(19) United States
(12) Patent Application Publication
(10) Pub. No.: US 2005/0043292 A1
(43) Pub. Date: Feb. 24, 2005

(54) Fluorinated lysine derivatives as dipeptidyl peptidase IV inhibitors

(75) Inventors: Janice C. Parker, Ledyard, CT (US); Bernard Hulin, Essex, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/923,146

(22) Filed: Aug. 19, 2004

Related U.S. Application Data
(60) Provisional application No. 60/496,969, filed on Aug. 20, 2003.

(51) Int. Cl. A61K 31/397; A61K 31/445

(52) U.S. Cl. 514/210.17; 514/330; 548/953; 546/245

(57) ABSTRACT

The invention relates to new therapeutically active and selective inhibitors of the enzyme dipeptidyl peptidase-IV (“DPP-IV”), pharmaceutical compositions comprising the compounds and the use of such compounds for treating diseases that are associated with proteins that are subject to processing by DPP-IV, such as Type 2 diabetes, metabolic syndrome (syndrome X or insulin resistance syndrome), hyperglycemia, impaired glucose tolerance, glucosuria, metabolic acidosis, arthritis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, short bowel syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome and to prevent disease progression in Type 2 diabetes. The invention also relates to a method of identifying an insulin secretagogue agent for diabetes.

### FIELD OF THE INVENTION

The present invention relates to new therapeutically active and selective inhibitors of the enzyme dipeptidyl peptidase-IV (hereinafter “DPP-IV”), pharmaceutical compositions comprising the compounds and the use of such compounds for treating diseases that are associated with proteins that are subject to processing by DPP-IV, such as Type 2 diabetes, metabolic syndrome (syndrome X or insulin resistance syndrome), hyperglycemia, impaired glucose tolerance, glucosuria, metabolic acidosis, arthritis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, short bowel syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome and to prevent disease progression in Type 2 diabetes. The invention also relates to a method of identifying an insulin secretagogue agent for diabetes.

### BACKGROUND OF THE INVENTION

Dipeptidyl peptidase-IV (EC 3.4.14.5) is a serine protease that preferentially hydrolyzes an N-terminal dipeptide from proteins having proline or alanine in the 2 position. The physiological roles of DPP-IV have not been fully elucidated, but it is believed to be involved in diabetes, glucose tolerance, obesity, appetite regulation, lipidemia, osteoporosis, neuropeptide metabolism and T-cell activation, among others.

DPP-IV has been implicated in the control of glucose homeostasis because its substrates include the incretin peptides glucagon-like peptide 1 (GLP-1) and gastric inhibitory polypeptide (GIP). Cleavage of the N-terminal amino acids from these peptides renders them functionally inactive. GLP-1 has been shown to be an effective anti-diabetic therapy in Type 2 diabetic patients and to reduce the meal-related insulin requirement in Type 1 diabetic patients. GLP-1 and/or GIP are believed to regulate satiety, lipidemia and osteogenesis. Exogenous GLP-1 has been proposed as a treatment for patients suffering from acute coronary syndrome, angina and ischemic heart disease.

Administration of DPP-IV inhibitors in vivo prevents N-terminal degradation of GLP-1 and GIP, resulting in higher circulating concentrations of these peptides, increased insulin secretion and improved glucose tolerance. On the basis of these observations, DPP-IV inhibitors are regarded as agents for the treatment of Type 2 diabetes, a disease in which glucose tolerance is impaired. In addition, treatment with DPP-IV inhibitors prevents degradation of Neuropeptide Y (NPY), a peptide associated with a variety of central nervous system disorders, and Peptide YY which has been linked to gastrointestinal conditions such as ulcers, irritable bowel disease and inflammatory bowel disease.

In spite of the early discovery of insulin and its subsequent widespread use in the treatment of diabetes, and the later discovery of and use of sulfonylureas (e.g. chlorpropamide, tolbutamide, acetohexamide), biguanides (e.g., phenformin, mefformin), and thiazolidinediones (e.g., rosiglitazone, pioglitazone) as oral hypoglycemic agents, the treatment of diabetes remains less than satisfactory.

The use of insulin, necessary in Type 1 diabetic patients and about 10% of Type 2 diabetic patients in whom currently available oral hypoglycemic agents are ineffective, requires multiple daily doses, usually by self-injection. Determination of the appropriate dosage of insulin necessitates frequent estimations of the glucose concentration in urine or blood. The administration of an excess dose of insulin causes hypoglycemia, with consequences ranging from mild abnormalities in blood glucose to coma, or even death.

Treatment of Type 2 diabetes usually comprises a combination of diet, exercise, oral agents, and in more severe cases, insulin. However, the clinically available hypoglycemics can have side effects which limit their use. A continuing need for hypoglycemic agents, which may have fewer side effects or succeed where others fail, is clearly evident.

Poorly controlled hyperglycemia is a direct cause of the multiplicity of complications (cataracts, neuropathy, nephropathy, retinopathy, cardiomyopathy) that characterize advanced Type 2 diabetes. In addition, Type 2 diabetes is a comorbid disease that frequently confounds hyperlipidemia, atherosclerosis and hypertension, adding significantly to the overall morbidity and mortality attributable to those diseases.

Epidemiological evidence has firmly established hyperlipidemia as a primary risk factor for cardiovascular disease (“CVD”) due to atherosclerosis. Atherosclerosis is recognized to be a leading cause of death in the United States and Western Europe. CVD is especially prevalent among diabetic subjects, at least in part because of the existence of multiple independent risk factors such as glucose intolerance, left ventricular hypertrophy and hypertension in this population. Successful treatment of hyperlipidemia in the general population, and in diabetic subjects in particular, is therefore of exceptional medical importance.

Hypertension (or high blood pressure) is a condition that can occur in many patients in whom the causative agent or disorder is unknown. Such “essential” hypertension is often associated with disorders such as obesity, diabetes and hypertriglyceridemia, and it is known that hypertension is positively associated with heart failure, renal failure and stroke. Hypertension can also contribute to the development of atherosclerosis and coronary disease. Hypertension, together with insulin resistance and hyperlipidemia, comprise the constellation of symptoms that characterize metabolic syndrome, also known as insulin resistance syndrome (“IRS”) and syndrome X.

Obesity is a well-known and common risk factor for the development of atherosclerosis, hypertension and diabetes. The incidence of obesity and hence of these diseases is increasing worldwide. Currently few pharmacological agents are available that reduce adiposity effectively and acceptably.

Osteoporosis is a progressive systemic disease characterized by low bone density and microarchitectural deterioration of bone tissue, with a consequent increase in bone fragility and susceptibility to fracture. Osteoporosis and the consequences of compromised bone strength are a significant cause of frailty, and of increased morbidity and mortality.

Heart disease is a major health problem throughout the world. Myocardial infarctions are a significant source of mortality among those individuals with heart disease. Acute coronary syndrome denotes patients who have or are at high risk of developing an acute myocardial infarction (MI).

Though there are therapies available for the treatment of diabetes, hyperglycemia, hyperlipidemia, hypertension, obesity and osteoporosis there is a continuing need for alternative and improved therapies.

Various indications for dipeptidyl peptidase inhibitors are discussed in the following review articles: Augustyns et al.: The unique properties of dipeptidyl-peptidase IV (DPP IV/CD26) and the therapeutic potential of DPP IV inhibitors, Curr. Medicinal Chem. 1999, 6, 311; Ohnuki et al.: Novel dipeptidyl peptidase IV inhibitors with antiarthritic effects, Drugs of the Future 1999, 24, 665-670; Villhauer et al.: DPP-IV inhibition and therapeutic potential, Annual Reports in Medicinal Chemistry 2001, 36, 191-200.

### SUMMARY OF INVENTION

This invention is directed to compounds of Formula Ia and Ib which are useful for the treatment of Type 2 diabetes, metabolic syndrome (also known as syndrome X and/or insulin resistance syndrome), hyperglycemia, impaired glucose tolerance, glucosuria, metabolic acidosis, arthritis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, short bowel syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome, and to prevent disease progression in Type 2 diabetes. The invention also relates to a method of identifying an insulin secretagogue agent for diabetes.

In one aspect, the invention is directed to a compound of Formula Ia, 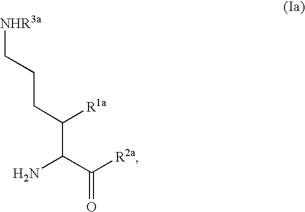
a a prodrug thereof, or a pharmaceutically acceptable salt of the compound or the prodrug, wherein:
R^{1a} is hydrogen, (C₁-C₈)alkyl, or (C₃-C₈)cycloalkyl;
R^{2a} is 3-fluoroazetidin-1-yl; 3,3-difluoroazetidin-1-yl; 3,4-difluoropyrrolidin-1-yl; 3,3,4-trifluoropyrrolidin-1-yl; 3,3,4,4-tetrafluoropyrrolidin-1-yl, 3-fluoropiperidin-1-yl; 4-fluoropiperidin-1-yl; 3,4-difluoropiperidin-1-yl; 3,3-difluoropiperidin-1-yl; 3,5-difluoropiperidin-1-yl; 3,4,5-trifluoropiperidin-1-yl; 3,3,4-trifluoropiperidin-1-yl; 3,3,5-trifluoropiperidin-1-yl; 3,4,4-trifluoropiperidin-1-yl; 3,3,4,4-tetrafluoropiperidin-1-yl; 3,3,4,5-tetrafluoropiperidin-1-yl; 3,3,5,5-tetrafluoropiperidin-1-yl; 3,3,4,4,5-pentafluoropiperidin-1-yl; 3,3,4,5,5-pentafluoropiperidin-1-yl; or 3,3,4,4,5,5-hexafluoropiperidin-1-yl;
R^{3a} is COR^{4a}, —COOR^{5a}, —CONR^{6a}R^{7a}, or —SO₂NR^{6a}R^{7a};
R^{4a} and R^{5a} are (A) (C₁-C₈)alkyl; (B) (C₃-C₈)cycloalkyl; (C) phenyl(C₀-C₈)alkyl; (D) phenoxy(C₁-C₈)alkyl; (E) a five- or six-membered unsaturated, partially saturated or saturated heterocyclyl(C₀-C₈)alkyl, said heterocyclyl comprising 1 to 3 of N, O, or S;
wherein said phenyl, phenoxy, and heterocyclyl are optionally and independently substituted with 1 to 3 of: (C₁-C₈)alkyl; (C₃-C₈)cycloalkyl; cyano; halo; (C₁-C₈)alkylsulfonyl; (C₁-C₈)alkylsulfonyloxy; phenyl(C₁-C₈)alkoxy; or phenyl optionally substituted with 1 to 3 of: (C₁-C₈)alkyl; halo; (C₁-C₈)alkoxy; cyano; hydroxy; trifluoromethyl; (C₁-C₈)alkylsulfonyl; (C₁-C₈)alkylsulfonyloxy; or phenyl(C₁-C₈)alkoxy; or,
(F) a nine- or ten-membered fused heterocyclyl, said fused heterocyclyl comprising 1 to 5 of: N, O, or S, and said fused heterocyclyl is optionally substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl, optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy;
R^{6a} and R^{7a} are taken separately and independently (A) hydrogen; (B) (C₁-C₈)alkyl; (C) (C₃-C₈)cycloalkyl; (D) phenyl(C₁-C₈)alkyl; (E) phenoxy(C₁-C₈)alkyl; or (F) a five- or six-membered unsaturated, partially saturated, or saturated heterocycl(C₀-C₈)alkyl, said heterocyclyl comprising 1 to 3 of N, O, or S;
wherein said phenyl, phenoxy, and heterocyclyl are optionally and independently substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy; or
R^{6a} and R^{7a} are taken together to form a 4- to 8-membered ring, whch is optionally substituted with (C₁-C₈)alkyl, (C₃-C₈)cycloalkyl, (C₁-C₈)alkoxy, phenyl, (C₁-C₈)alkoxy, or phenyl(C₁-C₈)alkyl.

In a preferred embodiment, R^{2a} is 3-fluoroazetidin-1-yl or 3-3-difluoroazetidin-1-yl.

In a more preferred embodiment, R^{2a} is 3,4-difluoropyrrolidin-1-yl or 3,3,4,4-tetrafluoropyrrolidin-1-yl.

In another preferred embodiment R^{3a} is COR^{4a}; R^{4a} is preferably:
a five- or six-membered unsaturated, partially saturated or saturated heterocycl(C₀-C₈)alkyl, said heterocyclyl comprising 1 to 3 of N, O, or S; and said heterocyclyl optionally substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl, optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy; or
a nine- or ten-membered fused heterocyclyl, said fused heterocyclyl comprising 1 to 5 of:N, O; or S, and said fused heterocyclyl is optionally substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl, optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy.

In a preferred embodiment, the compound of Formula Ia is:
(S)-Quinoxaline-2-carboxylic Acid [5-Amino-6-oxo-6-(3,3,4,4-tetrafluoro pyrrolidin-1-yl)-hexyl]-amide Hydrochloride
(S)-N-[5-Amino-6-oxo-6-(3,3,4,4-tetrafluoro-pyrrolidin-1-yl)-hexyl]-2-phenoxy-acetamide Hydrochloride;
(S)-Quinoxaline-2-carboxylic Acid [5-Amino-6-(3-fluoro-azetidin-1-yl)-6-oxo-hexyl]-amide Hydrochloride;
(S)-5-Methyl-2-phenyl-oxazole-4-carboxylic Acid [5-Amino-6-(3,3-difluoro-azetidin-1-yl)-6-oxo-hexyl]-amide Hydrochloride, or
(S)-3-Methyl-quinoxaline-2-carboxylic Acid [5-amino-6-oxo-6-(3,3,4,4-tetrafluoro-pyrrolidin-1-yl)-hexyl]-amide Hydrochloride. In another embodiment, the compound of Formula Ia may be any suitable salt, preferably any suitable anionic salt.

In another aspect, the invention is directed to a compound of Formula Ib 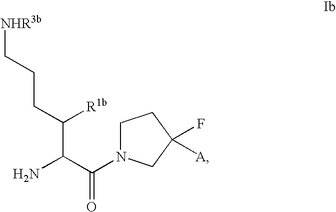
a prodrug thereof, or a pharmaceutically acceptable salt of the compound or the prodrug, wherein:
A is hydrogen or F;
R^{1b} is hydrogen, (C₁-C₈)alkyl, or (C₃-C₈)cycloalkyl;
R^{3b} is COR^{4b}, COOR^{5b}, CONR^{6b}R^{7b}, or SO₂NR^{6b}R^{7b};
R^{4b} and R^{5b} are (C₃-C₈)cycloalkyl, Het, or phenoxy(C₁-C₈)alkyl;
said phenoxy(C₁-C₈)alkyl includes a benzene ring optionally substituted with 1 to 3 of: (C₁-C₈)alkyl; (C₃-C₈)cycloalkyl; cyano; halo; (C₁-C₈)alkylsulfonyl; (C₁-C₈)alkylsulfonyloxy; phenyl(C₁-C₈)alkoxy; or phenyl, optionally substituted with 1 to 3 of (C₁-C₈)alkyl, halo, (C₁-C₈)alkoxy, cyano, hydroxy, trifluoromethyl, (C₁-C₈)alkylsulfonyl, (C₁-C₈)alkylsulfonyloxy, or phenyl(C₁-C₈)alkoxy; and,
said Het is selected from (I) furanyl, dihydrofuranyl, tetrahydrofuranyl, pyranyl, dihydropyranyl, tetrahydropyranyl, thienyl, dihydrothienyl, tetrahydrothienyl or a benzo-fused analogue of said Het; wherein each Het is optionally substituted on carbon or nitrogen with 1 to 3 of: (A) (C₃-C₈)cycloalkyl; (B) (C₁-C₈)alkylsulfonyl; (C) (C₁-C₈)alkylsulfonyloxy; (D) phenoxy(C₁-C₈)alkyl; wherein said phenoxy group comprises a phenyl ring optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; (E) phenyl, optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; (F) (C₁-C₈)alkyl, (G) cyano, (H) halo, (I) phenyl(C₁-C₈)alkoxy; or (J) trifluoromethyl; or
(II) pyridyl, pyridazinyl, pyrimidyl, pyrazinyl, imidazolyl, pyrazolyl, thiazolyl, isothiazolyl, oxazolyl, isoxazolyl, triazolyl, or a benzo-fused analogue of said Het; wherein each Het is independently substituted on carbon or nitrogen with 1 to 3 of: (A) (C₃-C₈)cycloalkyl; (B) (C₁-C₈)alkylsulfonyl; (C) (C₁-C₈)alkylsulfonyloxy; (D) phenoxy(C₁-C₈)alkyl; wherein said phenoxy group comprises a phenyl ring optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; (E) phenyl, optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; wherein Het is optionally substituted with 1 to 3 of (C₁-C₈)alkyl, cyano, halo, phenyl(C₁-C₈)alkoxy or trifluoromethyl; and
R^{6b} and R^{7b} are taken separately and independently and are (A) (C₃-C₈)cycloalkyl; (B) phenoxy(C₁-C₈)alkyl; (C) a five- or six-membered unsaturated, partially saturated or saturated heterocyclyl; wherein said heterocyclyl comprises 1 to 3 of: N; O; or S; and said heterocyclyl is substituted with 1 to 3 of (C₃-C₈)cycloalkyl, phenyl, (C₁-C₈)alkylsulfonyl, (C₁-C₈)alkylsulfonyloxy, or phenyl(C₁-C₈)alkoxy, or (D) a nine- or ten-membered fused heterocyclyl, wherein said fused heterocyclyl comprises 1 to 5 of, N, O, or S; and said fused heterocyclyl is substituted with 1 to 3 of, (C₃-C₈)cycloalkyl, phenyl, (C₁-C₈)alkylsulfonyl, (C₁-C₈)alkylsulfonyloxy, or phenyl(C₁-C₈)alkoxy.

The term “Formula I” as used herein, includes both Formula Ia and Formula Ib.

In a preferred embodiment, the compound of Formula I has an S configuration at the stereogenic carbon atom adjacent to the primary amine.

In another aspect, the invention is directed to a pharmaceutical composition comprising a therapeutically effective amount of a compound of Formula I, a prodrug thereof or a pharmaceutically acceptable salt of the compound or the prodrug, and optionally a pharmaceutically acceptable diluent or carrier. In one embodiment, the pharmaceutical composition comprises the compound of Formula I having an S configuration at the stereogenic carbon atom adjacent to the primary amine.

In a preferred embodiment, the pharmaceutical composition is of an amount effective in inhibiting the enzyme dipeptidyl peptidase-IV in a mammal.

In another preferred embodiment, the mammal is a human.

In another aspect, the invention is directed to a pharmaceutical composition which comprises a therapeutically effective amount of
a) a first compound of Formula I, a prodrug thereof or a pharmaceutically acceptable salt of the compound or the prodrug;
b) a second compound that is an antidiabetic agent selected from insulin and insulin analogs; insulinotropin; biguanides; α₂-antagonists and imidazolines; glitazones; aldose reductase inhibitors; glycogen phosphorylase inhibitors; sorbitol dehydrogenase inhibitors; fatty acid oxidation inhibitors; α-glucosidase inhibitors; β-agonists; phosphodiesterase inhibitors; lipid-lowering agents; antiobesity agents; vanadate and vanadium complexes and peroxovanadium complexes; amylin antagonists; glucagon antagonists; growth hormone secretagogues; gluconeogenesis inhibitors; somatostatin analogs; antilipotic agents; a prodrug of the antidiabetic agents, or a pharmaceutically acceptable salts of the antidiabetic agents and the prodrugs; and

In one embodiment, the composition further comprises a pharmaceutically acceptable carrier or diluent.

In another aspect, the invention is directed to a kit comprising:
a) a first dosage form comprising a compound of Formula I, a prodrug thereof or a pharmaceutically acceptable salt of the compound or the prodrug; and
b) a second dosage form comprising an antidiabetic agent selected from insulin and insulin analogs; insulinotropin; biguanides; α₂-antagonists and imidazolines; glitazones; aldose reductase inhibitors; glycogen phosphorylase inhibitors; sorbitol dehydrogenase inhibitors; fatty acid oxidation inhibitors; α-glucosidase inhibitors; β-agonists; phosphodiesterase inhibitors; lipid-lowering agents; antiobesity agents; vanadate and vanadium complexes and peroxovanadium complexes; amylin antagonists; glucagon antagonists; growth hormone secretagogues; gluconeogenesis inhibitors; somatostatin analogs; antilipolytic agents; prodrugs of the antidiabetic agents, or a pharmaceutically acceptable salts of the antidiabetic agents and the prodrug; and
c) a container for containing said first dosage (a) and said second dosage (b).

In a preferred embodiment of the kit, both the first and the second dosage forms independently comprise a pharmaceutically acceptable carrier or diluent.

In another aspect, the invention is directed to a therapeutic method of inhibiting dipeptidyl peptidase-IV comprising administering to a mammal in need of such treatment a therapeutically effective amount of a compound of Formula I, a prodrug thereof or a pharmaceutically acceptable salt of the compound or the prodrug, either alone or in combination with an antidiabetic agent as described above. In one embodiment, the method comprises administering a compound of Formula I having an S configuration at the stereogenic carbon atom adjacent to the primary amine.

In another aspect, the invention is directed to a method of treating a condition mediated by dipeptidyl peptidase-IV inhibition comprising administering to a mammal in need of such treatment a therapeutically effective amount of a compound of Formula I, a prodrug thereof or a pharmaceutically acceptable salt of the compound or the prodrug, either alone or in combination with an antidiabetic agent as described above. In one embodiment, the method comprises administering a compound of Formula I having an S configuration at the stereogenic carbon atom adjacent to the primary amine.

In one embodiment, the condition treated is Type 2 diabetes, metabolic syndrome (syndrome X or insulin resistance syndrome), hyperglycemia, impaired glucose tolerance, glucosuria, metabolic acidosis, arthritis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, short bowel syndrome, anxiety, depression, insomnia, chronic fatigue, epilepsy, eating disorders, chronic pain, alcohol addiction, diseases associated with intestinal motility, ulcers, irritable bowel syndrome, or inflammatory bowel syndrome.

In a preferred embodiment, the condition treated is Type 2 diabetes.

In another aspect, the invention is directed to a method of identifying an insulin secretagogue agent for diabetes, comprising:
a) administering an agent to a fasted, diabetic KK/H1J mouse; and
b) assessing a response in the mouse to a subsequent oral glucose challenge,
   wherein said agent is identified as a treatment for Type 2 diabetes, metabolic syndrome (syndrome X or insulin resistance syndrome), hyperglycemia, impaired glucose tolerance, glucosuria, metabolic acidosis, arthritis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, to prevent disease progression in Type 2 diabetes, anxiety, depression, insomnia, chronic fatigue, epilepsy, eating disorders, chronic pain, alcohol addiction, diseases associated with intestinal motility, ulcers, irritable bowel syndrome, inflammatory bowel syndrome or short bowel syndrome, if the mouse demonstrates an improvement in the symptoms.

In one embodiment of this aspect of the invention, the agent is a compound of Formula I.

The compounds and intermediates of the present invention may be named according to either the IUPAC (International Union for Pure and Applied Chemistry) or CAS (Chemical Abstracts Service, Columbus, Ohio) nomenclature systems.

The carbon atom content of the various hydrocarbon-containing moieties herein may be indicated by a prefix designating the minimum and maximum number of carbon atoms in the moiety, for example, the prefix (Cₐ-C_{b})alkyl indicates an alkyl moiety of the integer “a” to “b” carbon atoms, inclusive. Thus, for example, (C₁-C₆)alkyl refers to an alkyl group of one to six carbon atoms inclusive.

The term “alkoxy” refers to straight or branched, monovalent, saturated aliphatic chains of carbon atoms bonded to an oxygen atom that is attached to a core structure. Examples of alkoxy groups include methoxy, ethoxy, propoxy, butoxy, iso-butoxy, tert-butoxy, and the like.

The term “alkyl” as used herein, means a saturated monovalent straight or branched aliphatic hydrocarbon radical, wherein the number of carbon atoms may be defined in a parenthetical where the term is used. Examples of alkyl groups include methyl, ethyl, propyl, butyl, and the like.

The term “cycloalkyl” denotes a saturated monocyclic or bicyclic cycloalkyl group, optionally fused to an aromatic hydrocarbon group. Examples of cycloalkyl groups include cyclopropyl, cyclobutyl, cyclopentyl, cyclohexyl, cycloheptyl, indanyl, tetrahydronaphthyl, and the like.

The term “halogen” or “halo” represents chloro, bromo, fluoro, and iodo atoms and substituents.

The term “heterocyclyl” or “heterocycle” denotes a saturated monocyclic or polycyclic cycloalkyl group, in which at least one of the carbon atoms is replaced with a heteroatom such as nitrogen, oxygen, or sulfur. If the heterocyclyl contains more than one heteroatom, the heteroatoms may be the same or different.

A cyclic group may be bonded to another group in more than one way. If no particular bonding arrangement is specified, then all possible arrangements are intended. For example, the term “pyridyl” includes 2-, 3-, or 4-pyridyl.

The term “mammal” means animals including, for example, dogs, cats, cows, sheep, horses, and humans. Preferred mammals include humans.

The term “oxo”, means a carbonyl group formed by the combination of a carbon atom and an oxygen atom.

As used herein, the term “Formula I” includes Formula Ia and Formula Ib.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The expression “pharmaceutically acceptable salt” as used herein in relation to compounds of Formula I of this invention includes pharmaceutically acceptable anionic salts. The term “pharmaceutically acceptable anion” refers to a negative ion that is compatible chemically and/or toxicologically with the other ingredients of a pharmaceutical composition and/or the animal being treated therewith. Suitable anions include, but are not limited to, halides (e.g., chloride, iodide, and bromide), (C₁-C₁₂)alkylsulfonates (e.g., mesylate, ethylsulfonate, etc.), arylsulfonates (e.g., phenylsulfonate, tosylate, etc.), (C₁-C₁₂)alkylphosphonates, di(C₁-C₁₂)alkylphosphates (e.g., dimethylphosphate, diethylphosphate, α-diglycerol phosphate, etc.), arylphosphonates, arylphosphates, alkylarylphosphonates, alkylarylphosphates, (C₁-C₁₂)alkylcarboxylates (e.g., acetates, propionates, glutamates, glycerates, etc.), arylcarboxylates, and the like.

The compounds of the present invention may be isolated and used per se or in the form of their pharmaceutically acceptable salts, solvates and/or hydrates. The term “salts” refers to inorganic and organic salts of a compound of the present invention. These salts can be prepared in situ during the final isolation and purification of a compound, or by separately reacting the compound, or prodrug with a suitable organic or inorganic acid and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate and laurylsulphonate salts, and the like. See, e.g., Berge, et al., J. Pharm. Sci., 66, 1-19 (1977).

The term “prodrug” means a compound that is transformed in vivo to yield a compound of Formula (I) or a pharmaceutically acceptable salt, hydrate or solvate of the compound. Such compounds include, but are not limited to, N-acyl and N-carboalkoxy derivatives of Formula I compounds, as well as imine derivatives. The transformation may occur via various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The term “substituted” means that a hydrogen atom on a molecule has been replaced with a different atom or molecule. The atom or molecule replacing the hydrogen atom is denoted as a “substituent.”

The symbol “—” represents a covalent bond.

The phrase “reaction-inert solvent” or “inert solvent” refers to a solvent, or mixture of solvents, that does not interact with starting materials, reagents, intermediates, or products in a manner that adversely affects their desired properties.

The terms “treating”, “treated”, or “treatment” as employed herein includes preventative (e.g., prophylactic), palliative, or curative use or result.

Some of the compounds described herein contain at least one stereogenic center; consequently, those skilled in the art will appreciate that all stereoisomers (e.g., enantiomers and diasteroisomers, and racemic mixtures thereof) of the compounds illustrated and discussed herein are within the scope of the present invention. In particular, the carbon that is substituted with NH₂ in compounds of Formula I, and their intermediates, is stereogenic and these compounds are depicted and claimed in claim 1 and 8 as racemic mixtures and in claims 11 and 12 as the S enantiomers. One skilled in the art will also recognize that both the pyrrolidine and piperidine moieties of the compounds of Formula I may also contain at least one stereogenic center, such as, for example, where R² in Formula Ia is a fluorinated pyrrolidine or piperidine, or where A in Formula Ib is H. All stereoisomers (e.g., enantiomers and diasteroisomers, and racemic mixtures thereof) of these compounds claimed, illustrated and discussed herein are within the scope of the present invention.

Those skilled in the art will further recognize that the compounds of Formula I can exist in crystalline form as hydrates wherein molecules of water are incorporated within the crystal structure thereof and as solvates wherein molecules of a solvent are incorporated therein. All such hydrate and solvate forms are considered part of this invention.

This invention also includes isotopically-labeled compounds, which are identical to those described by Formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, sulfur and fluorine, such as ²H, ³H, ¹³C, ¹⁴C, ¹⁵N, ¹⁸O, ¹⁷O, ³⁵S, ³⁶Cl, ¹²⁵I, ¹²⁹I, and ¹⁸F respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of Formula I of this invention and prodrugs thereof can generally be prepared by carrying out the procedures disclosed in the schemes and/or in the Examples below, by substituting a readily available isotopically labeled reagent for a non-isotopically labeled reagent.

### DESCRIPTION OF INVENTION

In general, the compounds of Formula I of this invention may be prepared by methods that include processes known in the chemical arts, particularly in light of the description contained herein. Certain processes for the manufacture of the compounds of Formula I of this invention are illustrated by the following reaction schemes. Other processes are described in the experimental section. Some of the starting compounds for the reactions described in the schemes and Examples are prepared as illustrated herein. All other starting compounds may be obtained from general commercial sources, such as Sigma-Aldrich Corporation, St. Louis, Mo.

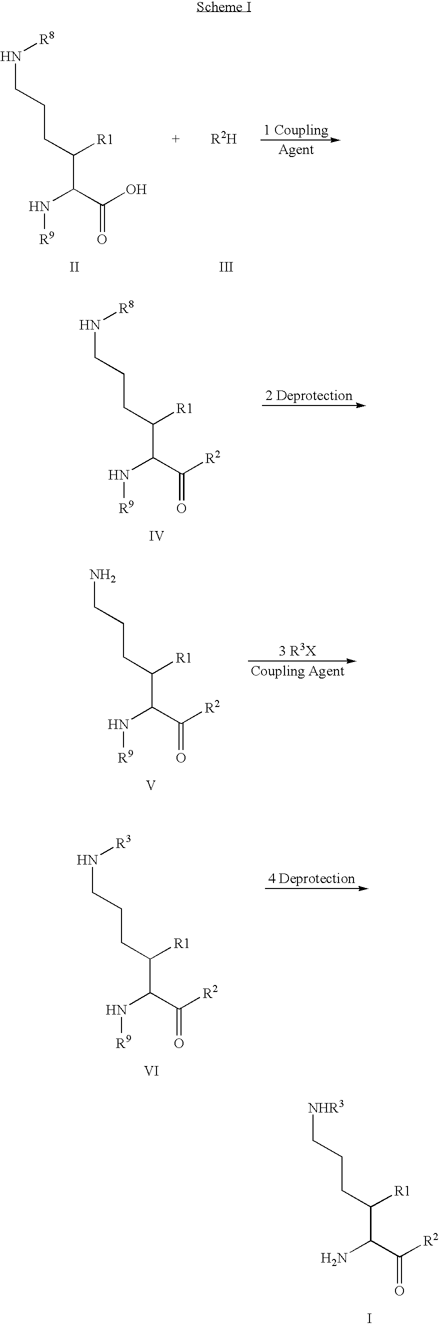

In Scheme I, R¹ refers to R^{1a} or R^{1b}; R³ refers to either R^{3a} or R^{3b}; and R² refers to R^{2a}, 3-fluoropyrrolidin-1-yl, or 3,3-difluoropyrrolidin-1-yl.

According to Scheme I, the compounds of Formula I, wherein R¹, R² and R³, are defined above, may be prepared by the synthetic sequence illustrated in Scheme I. Step 1 comprises coupling an amino acid compound of Formula II (e.g., (L)-Boc-ε-Cbz-lysine, (L)-Boc-ε-Cbz -β-methyl-lysine, wherein R⁸ and R⁹ are nitrogen-protecting groups, chosen so that R⁸ is inert to the conditions of step 1 while R⁹ is inert to the conditions of steps 1 to 3), with a compound of Formula III, preferably a fluorinated azetidine, pyrrolidine or piperidine, and more preferably, 3,3-difluoroazetidine hydrochloride or 3,3,4,4-tetrafluoropyrrolidine hydrochloride. A compound of Formula IV is produced.

Suitable nitrogen-protecting groups, R⁸ and R⁹ may independently include for example, but are not limited to, tert-butoxycarbonyl (“Boc”), benzyloxycarbonyl (“Cbz”), and fluorenylmethoxycarbonyl (“Fmoc”). Practitioners will appreciate that suitable protecting groups include those where R⁹ is stable to conditions which remove R⁸. Other examples of nitrogen-protecting groups are described in “Protective Groups in Organic Synthesis”, 2^{nd}. Ed., P. G. M. Wuts and T. W. Greene, p.315, incorporated herein by reference.

The coupling reaction described above is readily accomplished by dissolving a compound of Formula II and a compound of Formula III in a reaction inert solvent (e.g. dichloromethane) in the presence of base (e.g. triethylamine or pyridine). To the resulting solution is added a coupling agent (e.g. 1-(-3-dimethylaminopropyl)-3-ethylcarbodiimide hydrochloride) and an optional adjuvant (e.g. hydroxybenzotriazole). Other coupling agents may be utilized, such as dicyclohexylcarbodiimide, 2-ethoxy-1-ethoxycarbonyl-1,2-dihydroquinoline, carbonyldiimidazole or diethylphosphorylcyanide. The coupling is conducted in an inert solvent, preferably an aprotic solvent. Suitable solvents include, for example, acetonitrile, dichloromethane, dimethylformamide, and chloroform. For a discussion of other conditions useful for coupling carboxylic acids see Houben-Weyl, Vol XV, part II, E. Wunsch, Ed., G. Theime Verlag, 1974, Stuttgart, and those described in M. Bodansky, Principles of Peptide Synthesis, Springer-Verlag Berlin 1984, and The peptides. Analysis , Synthesis and Biology (ed. E. Gross and J. Meienhofer), vols 1-5 (Academic Press NY 1979-1983). The text of the above references is incorporated by reference.

The reaction is generally conducted at ambient pressure and temperature until the starting materials are no longer present as determined by thin layer chromatography or other analytical techniques well known to those skilled in the art. The coupled product of Formula IV may be isolated according to methods well known to those skilled in the art.

Deprotection (step 2) of a compound of Formula IV where R⁸ is benzyloxycarbonyl is performed by hydrogenolysis in the presence of 10% palladium or palladium hydroxide in ethanol or ethyl acetate at a suitable pressure, such as about 30 psi to about 60 psi, and preferably about 45 psi, for a period of time sufficient to bring the reaction to completion, usually overnight. Compound V is then isolated by filtration of the catalyst over diatomaceous earth and removal of the solvent.

In step 3 the compound of formula V is coupled with an appropriate reagent to give a compound of formula VI. If the coupling involves the formation of an amide bond, i.e. R³ is COR⁴, the coupling can be effected with an acid chloride R⁴COCl in the presence of a base such as triethylamine or diisopropylethylamine, or with the corresponding carboxylic acid R⁴COOH as described for Step 1. It will be understood by those skilled in the art that any carboxylic acid R⁴COOH which can be transformed into an acid chloride R⁴COCl can be coupled by this procedure to produce a compound of formula VI, with R⁴ being defined above as R^{4a} or R^{4b}. Accordingly, any acid R⁴COOH, wherein R⁴ includes an alkyl or cycloalkyl group, can be coupled using this procedure

Step 4, i.e. deprotection of the compound of formula VI is performed, if R⁹ is Boc, by dissolving a compound of Formula IV in an inert solvent (e.g. ethyl acetate) and cooling to about 0° C., followed by treatment with gaseous acid (e.g. hydrogen chloride) for about 1 minute. The reaction mixture is stirred for about 5 minutes to an hour and then allowed to reach room temperature, followed by stirring for about an additional 30 minutes to 16 hours. In one embodiment, the reaction mixture is stirred about 15 minutes, allowed to reach room temperature, then stirred an additional 30 minutes.

It will be obvious to one skilled in the art that in the sequence described above the identity of the protecting groups R⁸ and R⁹ is not limited to benzyloxycarbonyl and tert-butyloxycarbonyl. Different groups may be used. In this regard, it is recommended to adjust the deprotection conditions to match the particular protecting groups chosen.

The compounds described herein contain at least one stereogenic center; consequently, those skilled in the art will appreciate that all stereoisomers (e.g., enantiomers and diastereoisomers, and racemic mixtures thereof) of the compounds illustrated and discussed herein are within the scope of the present invention. In particular, the protected starting amino acid, Formula II, which is depicted as having the L configuration in Scheme I, may be a mixture of D and L isomers. Consequently, the compounds of Formula I may exist as DL mixtures and theses mixtures are within the scope of this invention. One skilled in the art will also recognize that the carbon atom bearing R¹ may be stereogenic if R¹ is not hydrogen, and that both the pyrrolidine or piperidine moieties of Formula Ia or Ib may also contain at least one stereogenic center. All stereoisomers (e.g., enantiomers and diastereoisomers, and racemic mixtures thereof) of these compounds claimed, illustrated and discussed herein are within the scope of the present invention.

The optically active amino acids may be obtained by resolution or by asymmetric synthesis or by other methods well known to those skilled in the art, prior to coupling in Step 1 of Scheme I. Alternatively, resolution, if so desired, may be accomplished at a later point in the synthesis of the compounds of Formula I by techniques known to those of ordinary skill in the art.

Compound III of Scheme I may be prepared as known to those of ordinary skill in the art. For example, 3,3-difluoropyrrolidine hydrochloride may be prepared as described by Giardina, G et al. Synlett. 1995, 55. 3,3,4,4-Tetrafluoropyrrolidine may be prepared as described in Chaudry et al. J. Chem. Soc., 1964, 874. Alternatively, 3,3,4,4-tetrafluoropyrrolidine may be prepared as depicted below in Scheme II.

As used herein, the term “inert solvent” is a solvent whose structure does not contain functional groups likely to interfere with the reaction. Examples include dichloromethane, 1,2-dichloroethane, tetrahydrofuran (THF), and dimethylformamide (DMF)

As used herein, the term “activating reagent” is one that transforms a hydroxyl group into a leaving group such as bromide, iodide, alkylsulfonate or arylsulfonate.

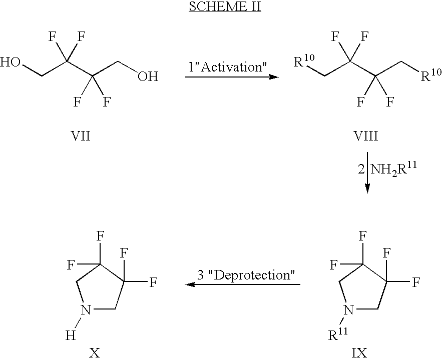

According to Scheme II, in Step 1, the hydroxyl groups of 2,2,3,3-tetrafluorobutanediol (VII) are activated to a leaving group, R¹⁰, (wherein R¹⁰ may be Br, I or SO₃R¹², wherein R¹² may either be (1) a C₁-C₈ straight or branched alkyl, optionally substituted with one or more fluorines, or (2) an aryl group, optionally substituted with halogen or a C₁-C₈ straight or branched alkyl optionally substituted with one to four fluorines). Preferably, however, R¹⁰ is a trifluoromethylsulfonyloxy radical (F₃CSO₃). The activating reagents necessary to transform the alcohol function to, for example, bromine or iodine are well-known to those skilled in the art (see e.g. March, Advanced Organic Chemistry, 3^{rd} ed. pp. 382-384 and Larock, Comprehensive Organic Transformations, pp. 353-360, incorporated by reference) and include, but are not limited to, the following activating reagents: hydrogen bromide (“HBr”), phosphorus tribromide (“PBr₃”), phosphorus pentabromide (“PBr₅”), thionyl bromide (“SOBr₂”) and hydrogen iodide (“HI”). Preferred activating reagent combinations are triphenylphosphine/carbon tetrabromide (“Ph₃P/CBr₄”), Ph₃P/N-bromosuccinimide, potassium iodide/phosphoric acid (“KI/H₃PO₄”), Ph₃P/I₂ and Me₃SiCl/NaI. Activation of the alcohol function to an alkyl or arylsulfonate is accomplished by reaction with the corresponding sulfonyl chloride or sulfonic anyhydride in an inert solvent in the presence of a base, such as pyridine or triethylamine, which is cooled (0° C.) and stirred for about one hour. The reaction mixture is then stirred at room temperature for about an additional hour.

In Step 2, the reaction of compound VIII with a primary amine R¹¹NH₂, wherein R¹¹ is an alkyl or aryl “protecting” group (i.e., such that the bond linking R¹¹ to the nitrogen atom may be easily cleaved by standard chemical manipulation known to those skilled in the art in Step 3), is accomplished by heating the solution under reflux overnight. Examples of N-protecting groups are described in “Protective Groups in Organic Synthesis”, 2^{nd}. Ed., P. G. M. Wuts and T. W. Greene, p.362, incorporated herein by reference, and include, for example, benzyl, tert-butyl, allyl and benzhydryl. Preferably, R¹¹ is benzyl, in which case the deprotection, Step 3, is performed by hydrogenolysis in the presence of palladium.

Step 3 can be performed on a cationic salt of intermediate compound IX, such as hydrochloride, hydrobromide, acetate, trifluoroacetate etc., in an appropriate solvent (e.g. water, methanol or ethanol). Removal of the R¹¹ protecting group from compound IX may be accomplished under conditions appropriate for the particular R¹¹ protecting group in use. Such conditions include, for example, (a) hydrogenolysis where R¹¹ is benzyl; (b) treatment with a strong acid, such as trifluoroacetic acid or hydrochloric acid, wherein R¹¹ is tert-butyl; or (c) treatment with tributyltinhydride and acetic acid in the presence of catalytic bis(triphenylphosphine) palladium (II) chloride where R¹¹ is allyl.

If R¹¹ is benzyl, deprotection is performed by hydrogenolysis in the presence of 10% palladium in ethanol at about 45 psi for about 3 hours. The final compound X is, thus, isolated as the corresponding cationic salt by filtration of the catalyst over diatomaceous earth, removal of the solvent and trituration with a non-hydroxylic solvent, such as diethyl ether, diisopropyl ether, ethyl acetate, 1,4-dioxane or tetrahydrofuran. Compound X is utilized, as described above in Scheme I, for preparation of DPP-IV inhibitor compounds of Formula Ia.

The compounds of Formula I of the present invention are useful for the treatment of dipeptidyl peptidase-IV related conditions; the treatment of Type 2 diabetes; the prevention of disease progression in Type 2 diabetes; the treatment of Type 1 diabetes, impaired glucose tolerance, hyperglycemia, metabolic syndrome (syndrome X and/or insulin resistance syndrome), glucosuria, metabolic acidosis, arthritis, cataracts, diabetic neuropathy, diabetic nephropathy, diabetic retinopathy, diabetic cardiomyopathy, obesity, conditions exacerbated by obesity, the treatment of hypertension, hyperlipidemia, atherosclerosis, osteoporosis, osteopenia, frailty, bone loss, bone fracture, acute coronary syndrome, short stature due to growth hormone deficiency, infertility due to polycystic ovary syndrome, anxiety, depression, insomnia, chronic fatigue, epilepsy, eating disorders, chronic pain, alcohol addiction, diseases associated with intestinal motility, ulcers, irritable bowel syndrome, inflammatory bowel syndrome and short bowel syndrome.

The present invention also relates to therapeutic methods for treating or preventing the above described conditions in a mammal, including a human, wherein a compound of Formula I of this invention is administered as part of an appropriate dosage regimen designed to obtain the benefits of the therapy. The appropriate dosage regimen, the amount of each dose administered and the intervals between doses of the compound will depend upon the compound of Formula I of this invention being used, the type of pharmaceutical compositions being used, the characteristics of the subject being treated and the severity of the conditions.

In general, an effective dosage for the compounds of Formula I described above is in the range of 0.01 mg/kg/day to 30 mg/kg/day, preferably 0.01 mg/kg/day to 1 mg/kg/day in single or divided doses. Some variation in dosage will necessarily occur, however, depending on the condition of the subject being treated. The individual responsible for dosing will, in any event, determine the appropriate dose for the individual subject. Practitioners will appreciate that “kg” refers to the weight of the patient measured in kilograms.

The compounds of Formula I of this invention may be administered to a subject in need of treatment by a variety of conventional routes of administration, including orally and parenterally, (e.g., intravenously, subcutaneously or intramedullary). Further, the pharmaceutical compositions of this invention may be administered intranasally, as a suppository, or using a “flash” formulation, i.e., allowing the medication to dissolve in the mouth without the need to use water.

The compounds of this invention may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like.

Thus, for purposes of oral administration, tablets containing various excipients such as sodium citrate, calcium carbonate and/or calcium phosphate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

Methods of preparing various pharmaceutical compositions with a certain amount of active ingredient are known, or will be apparent in light of this disclosure, to those skilled in this art. For examples of methods of preparing pharmaceutical compositions, see Reminqton's Pharmaceutical Sciences, Mack Publishing Company, Easton, Pa., 19th Edition (1995).

### Biological Protocols

The utility of the compounds of Formula I, the stereoisomers and prodrugs thereof, and the pharmaceutically acceptable salts of the compounds, stereoisomers, and prodrugs, in the treatment or prevention of diseases (such as are detailed herein) in animals, particularly mammals (e.g., humans) may be demonstrated by the activity thereof in conventional assays known to one of ordinary skill in the relevant art, including the in vitro and in vivo assays described below. Such assays also provide a means whereby the activities of the compounds of Formula I can be compared with the activities of other known compounds.

### In vitro Assay for Dipeptidyl Peptidase Inhibition

The dipeptidyl peptidase inhibition may be demonstrated in vitro by the following assay, which is adapted from published methods for the measurement of DPP-IV activity (Assay of dipeptidyl peptidase IV in serum by fluorimetry of 4-methoxy-2-naphthylamide. (1988) Scharpe, S., DeMeester, I., Vanhoof, G., Hendriks, D., Van Sande, M., Van Camp, K. and Yaron, A. Clin. Chem. 34:2299-2301; Dipeptidyl peptidases of human lymphocytes (1988) Lodja, Z. Czechoslovak Medicine, 11: 181-194.). 150 μL of an enzyme-substrate solution is pipetted into microtiter wells of a polystyrene 96-well plate, and maintained at 4° C. The enzyme-substrate solution comprises 50 μM Gly-Pro4-methoxy B naphthylamide HCl in 50 mM Tris assay buffer pH 7.3 containing 0.1M sodium chloride, 0.1% (v/v) Triton and 50 μU/mL DPP-IV (Enzyme Systems Products Cat#SPE-01, DPP-IV 5 mU/mL stock). 5μL/well of compounds of Formula I are added, bringing the final compound of Formula I concentrations to 3 μM-10 nM per well.

Controls. Enzyme is omitted from four (4) wells, as a reagent blank. 5 μL of 3 mM Diprotin A is added to four wells as a positive quality control, providing a final Diprotin A concentration of 100 μM. To measure total enzyme activity (i.e. a negative control), without the influence of any compounds of Formula I, 5 μL of distilled water is added to four wells.

The entire assay is incubated overnight (about 14-18 hours) at 37° C. The reaction is quenched by adding 10 μL of Fast Blue B solution (0.5 mg/mL Fast Blue B in a buffer comprising 0.1 M sodium acetate pH 4.2 and 10% (v/v) Triton X-100 to each well, followed by shaking for approximately 5 minutes at room temperature. The plates may be analyzed on a Spectramax spectrophotometer, or equivalent equipment, (absorption maximum at 525 nm). IC₅₀ data for compounds may be obtained by measuring the activity of DPP-IV over a range of compound concentrations from 10 nM to 3 μM.

### In vivo Assay for Glucose Lowering

The glucose lowering effects of DPP-IV inhibitors, such as compounds of Formula I, may be exemplified in 4-6 week old KK/H1J mice (Jackson Labs) in the context of an oral glucose tolerance test.

Oral glucose tolerance tests (“OGTT”) have been in use in humans since, at least, the 1930s, Pincus et al., Am. J. Med. Sci, 188: 782 (1934), and are routinely used in the diagnosis of human diabetes, though not to evaluate the efficacy of therapeutic agents in patients.

KK mice have been used to evaluate glitazones (Fujita et al. Diabetes 32:804-810 (1983); Fujiwara et al., Diabetes 37: 154948 (1988); Izumi et al. Biopharm Durg. Dispos. 18:247-257 (1997)), metformin (Reddi et al. Diabet. Metabl. 19:44-51 (1993)), glucosidase inhibitors (Hamada et al. Jap. Pharmacol. Ther. 17:17-28 (1988); Matsuo et al. Am. J. Clin. Nutr. 55:314S-317S (1992)), and the extra-pancreatic effects of sulfonylureas (Kameda et al Arzenim. Forsch./Drug Res. 32:39044 (1982); Muller et al. Horm. Metabl. Res. 28:469-487 (199)).

KK mice are derived from an inbred line first established by Kondo et al. (Kondo et al. Bull. Exp. Anim. 6:107-112 (1957)). The mice spontaneously develop a hereditary form of polygenic diabetes that progresses to cause renal, retinal and neurological complications analogous to those seen in human diabetic subjects, but they do not require insulin or other medication for survival.

Another aspect of the invention is directed to the use of KK mice to evaluate the effects of insulin secretagogue agents in the context of an oral glucose tolerance test.

The mice are fasted overnight (about 14-18 hours), but allowed free access to water. After fasting, (time “t”=0), 25 μL of blood is drawn from the retro-orbital sinus and added to 0.025% heparinized saline (100 μL) on ice. The mice (10 per group) are then orally dosed with a solution of a compound of Formula I in 0.5% methylcellulose (0.2 mL/mouse). Two controls groups receive only 0.5% methylcellulose. At t=15 minutes, the mice are bled, as described above, and then dosed with 1 mg/kg glucose in distilled water (0.2 mL/mouse). The first control group is dosed with glucose. The second control group is dosed with water. At t=45 minutes, the mice are again bled, as described above. The blood samples are centrifuged, the plasma collected and analyzed for glucose content on a Roche-Hitachi 912 glucose analyzer. The data may be expressed as percent (%) inhibition of glucose excursion relative to the two control groups (i.e. the glucose level in the animals receiving glucose but no test compound representing 0% inhibition and the glucose concentration in the animals receiving only water representing 100% inhibition).

### General Experimental Procedures

Melting points were determined on a capillary melting point apparatus by Thomas Scientific (Swedesboro, N.J.), and are uncorrected.

Flash chromatography was performed according to the method described by W. C. Still et al. in J. Org. Chem. 1978, 43, 2923.

Hydrogenations were performed in a Parr (Moline, Ill.) 3911 shaker type hydrogenation apparatus (hereafter referred to as a Parr hydrogenator) at the pressures indicated.

NMR chemical shifts are given in parts per million downfield from tetramethylsilane (for proton) or fluorotrichloromethane (for fluorine). Spectra were recorded on a Varian (Palo Alto, Calif.) Unity 400 MHz spectrometer. Mass Spectra were recorded on a Waters (Milford, Mass.) Micromass Platform II spectrometer.

The following enumerated Preparations describe the preparation of certain intermediates used in the Examples below. The Preparations and Examples below are intended to illustrate particular embodiments of the invention and preparations thereto and are not intended to limit the specification, including the claims, in any manner. The compounds exemplified hereinafter, Examples 1-43, displayed in vitro activity with an IC₅₀ (concentration of test compound required for 50% inhibition) range of about 0.017 μM to 1.4 μM. In particular, the compound of Example 6 exhibited about an IC₅₀ of 1.4 μM.

Unless noted otherwise, all reactants were obtained commercially. Unless indicated otherwise, the following abbreviations have the indicated meanings:
br—broad peaks
° C.—degree Celsius
CDCl₃—deuterated chloroform
CD₃OD—deuterated methanol
d—doublet peak
dd—double doublet peak
D₂O—deuterium oxide
DMF—dimethylformamide
DMSO—dimethyl sulfoxide
DMSO-d₆—perdeuterated dimethyl sulfoxide
dt—double triplet peak
g—gram(s)
H (e.g., 1H, 2H)—hydrogen(s)
Hz—hertz
J—coupling constant
m—multiplet
mg—milligram(s)
MHz—megahertz
mL—milliliter(s)
mmol—milimole(s)
mp—melting point
N—normality (e.g., 1 N sodium hydroxide)
NMR—nuclear magnetic resonance
psi—pounds per square inch (1 psi=6894.76 Pa)
q—quartet peak
s—singlet peak
t—triplet peak
td—triple doublet peak
μL-microliter

### Preparation 1 (S)-[5-Amino-1-(3,3,4,4-tetrafluoro-pyrrolidine-1-carbonyl)-pentyl]-carbamic Acid tert-Butyl ester

Step 1: Trifluoro-methanesulfonic Acid 2,2,3,3-Tetrafluoro-4-(trifluoro-methanesulfonyloxy)-butyl Ester.

To a cooled (about 0° C.) solution of 2,2,3,3-tetrafluorobutanediol (15 grams, 93 mmol) and pyridine (19 mL, 230 mmol) in dichloromethane (250 mL), was added dropwise trifluoromethanesulfonic anhydride (34 mL, 200 mL). After the addition, the mixture was stirred at about 0° C. for about one hour, followed by stirring at room temperature for one additional hour, then diluted with dichloromethane, washed with water and brine, dried over magnesium sulfate, filtered and concentrated to near dryness, leaving a dichloromethane-containing oil. ¹H NMR (400 MHz, CDCl₃) δ 4.82 (m). ¹⁹F NMR (376 MHz, CDCl₃) δ −120.83 (m, 4H), −74.38 (s, 6H).

Step 2: 1-Benzyl-3,3,4,4-tetrafluoro-pyrrolidine Hydrochloride

A solution of the crude trifluoro-methanesulfonic acid, 2,2,3,3-tetrafluoro-4-(trifluoro-methanesulfonyloxy)-butyl ester, benzylamine (10 mL, 93 mmol) and triethylamine (33 mL, 230 mmol) in ethanol (230 mL) was heated to reflux overnight. The mixture was concentrated to about one-third of its volume, diluted with ether, washed with 1 N sodium hydroxide, water and brine, dried over magnesium sulfate, filtered and concentrated to an oil. The oil was redissolved in ether, cooled to about 0° C. and saturated with hydrogen chloride. The precipitate was collected and dried (23.8 grams, 95%, (mp: 139-143° C.). ¹H NMR (400 MHz, D₂O) δ 4.05 (m, 4H), 4.41 (s, 2H), 7.26-7.41 (m, 5H), ¹⁹F NMR (376 MHz, D₂O) δ −118.42 (t, J=13.2 Hz).

Step 3: 3,3,4,4-Tetrafluoro-pyrrolidine Hydrochloride.

A solution of 1-benzyl-3,3,4,4-tetrafluoro-pyrrolidine hydrochloride (23.8 grams, 88 mmol) in ethanol (300 mL) containing 10% palladium on carbon was treated with hydrogen in a Parr hydrogenator at 45 psi for 3 hours. The reaction mixture was filtered through diatomaceous earth. The filtrate was concentrated to dryness. The residue was triturated with ether and the solid was collected and dried (13.4 grams, 85%, (melting point: 193-196° C.). ¹H NMR (400 MHz, D₂O) δ 3.93 (m, 4H), 7.26-7.41 (m, 5H). ¹⁹F NMR (376 MHz, D₂O) δ −122.25 (m)

Step 4: (S)-[5-Benzyloxycarbonylamino-1-(3,3,4,4-tetrafluoro-pyrrolidine-1-carbonyl)-pentyl]-carbamic Acid tert-Butyl ester

To a mixture of (L)-Nδ-(tert-butoxycarbonyl)-Nε-(carbobenzyloxy)-L-lysine (5 g, 13 mmol), 3,3,4,4-tetrafluoro-pyrrolidine hydrochloride (2.59 g, 14 mmol), hydroxybenzotriazole (2.3 g, 179 mmol) and triethylamine (2.4 mL, 17 mmol) in dichloromethane (75 mL) was added 1-(-3-dimethylaminopropyl)-3-ethylcarbodiimide hydrochloride (3.8 g, 20 mmol). The mixture was stirred at room temperature overnight, diluted with dichloromethane, washed with 1 N sodium hydroxide, 1 N hydrochloric acid, water and brine, dried over magnesium sulfate and concentrated. The product was purified by flash-chromatography (30% acetone/hexanes) and isolated as an oil (4.1 g, 62%).

Step 5: (S)-[5-Amino-1-(3,3,4,4-tetrafluoro-pyrrolidine-1-carbonyl)-pentyl]-carbamic Acid tert-Butyl Ester

20% Palladium hydroxide on carbon (0.40 g, 0.57 mmol) was added to a solution of (S)-[5-benzyloxycarbonylamino-1-(3,3,4,4-tetrafluoro-pyrrolidine-1-carbonyl)-pentyl]-carbamic acid tert-butyl ester (4.1 g, 8.1 mmol) in ethanol (100 mL) and the mixture was hydrogenated at 45 psi overnight in a Parr hydrogenator. The reaction mixture was filtered through diatomaceous earth and concentrated in vacuo to an oil (3.0 g, 100%). ¹H NMR (400 MHz, CD₃OD) δ 1.30-1.70 (m, 6H), 1.41 (s, 9H), 2.71 (t, J=7.27 Hz, 2H), 3.97 (m, 2H), 4.18-4.27 (m, 2H), 4.43 (q, J=12.6 Hz, 1H).

### Preparation 2 (S)-[5-Amino-1-(3,3,difluoro-pyrrolidine-1-carbonyl)-pentyl]-carbamic Acid tert-Butyl Ester

(S)-[5-Amino-1-(3,3,difluoro-pyrrolidine-1-carbonyl)-pentyl]-carbamic acid tert-butyl ester was prepared by coupling 3,3-difluoropyrrolidine hydrochloride (prepared according to Giardina, G. et al, Synlett 1995, 55) and (L)-Nα-(tert-butoxycarbonyl)-Nε-(carbobenzyloxy)-L-lysine followed by hydrogenolysis of the carbobenzyloxy group, as described in Preparation 1, Steps 4 and 5. ¹H NMR (400 MHz, CD₃OD) δ 1.36-1.71 (m, 6H), 1.42 (s, 9H), 2.33-2.53 (m, 2H), 2.72 (t, J=7.27 Hz, 2H), 3.57-4.32 (m, 5H).

### Preparation 3 (S)-[5-Amino-1-(3-fluoro-azetidine-1-carbonyl)-pentyl]-carbamic Acid tert-Butyl Ester

Step 1: 1-Benzhydryl-3-fluoro-azetidine

Diethylaminosulfur trifluoride (10 g, 63 mmol) was added to a solution of 1-benzhydrylazetidin-3-ol (5 g, 21 mmol). The mixture was stirred overnight then poured into ice-water and extracted with ethyl acetate. The combined extracts were washed with brine, dried over magnesium sulfate and concentrated. The residue was triturated with acetone and dried, giving the product as a colorless solid (2.65 g, 52%, mp 154-156° C.).

Step 2: 3-Fluoro-azetidine Hydrochloride

1-Benzhydryl-3-fluoro-azetidine (2.6 g, 10.8 mmol) and 20% palladium hydroxide (0.6 g, 0.86 mmol) were added to a solution of hydrogen chloride (2.5 g) in methanol (70 mL). The mixture was hydrogenated at 50 psi in a Parr hydrogenator. After 16 hours an additional portion of 20% palladium hydroxide (0.6 g, 0.86 mmol) was added and the mixture was again hydrogenated overnight. The reaction mixture was filtered through diatomaceous earth and concentrated to a solid which was triturated with hexanes and dried overnight (1.0 g, 83%).

Step 3: (S)-[5-Amino-1-(3-fluoro-azetidine-1-carbonyl)-pentyl]-carbamic Acid tert-Butyl Ester was prepared by coupling 3-fluoroazetidine hydrochloride and (L)-Nδ-(tert-butoxycarbonyl)-Nδ-(carbobenzyloxy)-L-lysine followed by hydrogenolysis of the benzyloxycarbonyl group, as described in Preparation 1, Steps 4 and 5. ¹H NMR (400 MHz, CD₃OD) δ1.35-1.66 (m, 6H), 1.42 (s, 9H), 2.74 (m, 2H), 3.90-4.15 (m, 2H), 4.20-4.78 (m, 3H), 5.25 (m, 0.5H), 5.44 (m, 0.5H).

### Preparation 4 (S)-[5Amino-1-(3,3-difluoro-azetidine-1-carbonyl)-pentyl]-carbamic Acid tert-Butyl Ester

(S)-[5-Amino-1-(3,3,difluoro-azetidine-1-carbonyl)-pentyl]-carbamic acid tert-butyl ester was prepared by coupling 3,3-difluoroazetidine hydrochloride (prepared according to Carson, W. R. et al, W00047582) and (L)-Nδ-(tert-butoxycarbonyl)-Nδ-(carbobenzyloxy)-L-lysine followed by hydrogenolysis of the carbobenzyloxy group, as described in Preparation 1, Steps 4 and 5. ¹H NMR (400 MHz, CD₃OD) δ1.34-1.73 (m, 6H), 1.42 (s, 9H), 2.69 (t, J=7.3 Hz, 2H), 4.03 (dd, J=8.6, 5.8 Hz, 1H), 4.32 (m, 2H), 4.60 (q, J=11.5 Hz, 1H), 4.78 (q, J=11.5 Hz, 1H).

### Preparation 5 3-Methyl-quinoxaline-2-carbonyl Chloride

3-Methyl-quinoxaline-2-carboxylic acid methyl ester (0.20 g, 0.99 mmol) was dissolved in methanol (8 mL) and 2 N sodium hydroxide (2 mL). The mixture was stirred for 30 minutes, then concentrated to about ⅓ of its volume, acidified with 1 N hydrochloric acid and extracted with ethyl acetate. The combined extracts were washed with water and brine, dried over magnesium sulfate and concentrated to a solid which was dried on high vacuum (0.16 g, 87%).

The solid (0.10 g, 0.53 mmol) was slurried in dichloromethane and oxalyl chloride (56 μL, 0.64 mmol) was added at 0° C. The mixture was warmed to room temperature and stirred for 1 hour, then concentrated to a tan solid (62 mg, 57%).

### Preparation 6 2-(4-Methoxy-phenyl)-5-methyl-oxazole-4-carbonyl Chloride

Step 1: (±)-2-Amino-3-oxo-butyric acid methyl ester hydrochloride.

10% palladium on carbon (0.6 g, 0.57 mmol) was added to a solution of 2-hydroxyimino-3-oxo-butyric acid methyl ester (Corven et al. J. Am. Chem. Soc. 1944, 66, 1150) (6.2 g, 43 mmol) in methanol (70 mL) containing hydrogen chloride (8 g). The mixture was hydrogenated on a Hydrogenator at 45 psi overnight. The catalyst was filtered over diatomaceous earth and the filtrate was concentrated, leaving a colorless solid (5.4 g, 84%).

Step 2: 2-(4-Methoxy-benzoylamino)-3-oxo-butyric acid Ethyl Ester.

To a suspension of 2-amino-3-oxo-butyric acid ethyl ester (Soukup et al. Helv. Chim. Acta 1987, 70, 232) (10 g, 55 mmol) in chloroform (200 mL), cooled to 0° C., was added p-anisoyl chloride (10.2 g, 60 mmol), followed by triethylamine (11.6 g, 115 mmol), the latter over 15 minutes. The reaction was stirred for 30 minutes at room temperature, then washed with water and brine, dried over magnesium sulfate and concentrated. The residue was purified by filtration through a plug of silica gel (500 g), using ethyl acetate/hexanes (1:1) as the eluant, to give the product, 2-(4-methoxy-benzoylamino)-3-oxo-butyric acid ethyl ester, as a solid (14.8 g, 96%, mp 52-55° C.).

Step 3: 2-(4-Methoxy-phenyl)-5-methyl-oxazole-4-carboxylic Acid Ethyl Ester

The solid (14.8 g, 53 mmol) was combined with phosphorous oxychloride (150 mL) and the mixture was heated under reflux for 1.5 hours then cooled and concentrated to a brown syrup which was dissolved in dichloromethane (250 mL). The solution was washed with water, saturated sodium bicarbonate, water and brine, dried over magnesium sulfate and concentrated to give the product, 2-(4-methoxy-phenyl)-5-methyl-oxazole-4-carboxylic acid ethyl ester, as a tan solid (12.8 g, 92%, mp 56-57° C.).

Step 4: 2-(4-Methoxy-phenyl)-5-methyl-oxazole-4-carboxylic Acid

(4-Methoxy-phenyl)-5-methyl-oxazole-4-carboxylic acid ethyl ester (100 mg, 0.38 mmol) was dissolved in a mixture of ethanol (5 mL) and 2 N sodium hydroxide (1 mL). The mixture was stirred for 30 minutes, concentrated to about one third of its volume and acidified with 1 N hydrochloric acid. The precipitate was filtered, washed with water and dried on high vacuum overnight (77 mg, 87%).

Step 5: 2-(4-Methoxy-phenyl)-5-methyl-oxazole-4-carboxylic Acid Chloride

To a suspension of the acid (77 mg, 0.33 mmol) in dichloromethane (2 mL), cooled to 0° C., was added oxalyl chloride (35 μL, 0.40 mmol), followed by dimethylformamide (1 drop). The reaction was stirred at room temperature for 1 hour then concentrated in vacuo, leaving 2-(4-methoxy-phenyl)-5-methyl-oxazole-4-carbonyl chloride as a pink solid (80 mg, 96%, mp 89° C.)

### Preparation 7 2-Cyclohexyl-5-methyl-oxazole-4-carbonyl Chloride

Step 1: (±)-2-(Cyclohexanecarbonyl-amino)-3-oxo-butyric Acid Methyl Ester

To a suspension of (±)-2-amino-3-oxo-butyric acid ethyl ester hydrochloride (5.4 g, 32 mmol) and triethylamine (10.4 mL, 74 mmol) in dichloromethane (150 mL), cooled to 0° C., was added dropwise a solution of cyclohexanecarbonyl chloride (4.3 mL, 32 mmol) in dichloromethane (50 mL). The mixture was warmed to room temperature and after 1 hour dimethylformamide (10 mL) was added. After an additional 4 hours, the homogeneous mixture was diluted with dichloromethane, washed with 1 N hydrochloric acid, water and brine, dried over magnesium sulfate and concentrated. The product was purified by flash-chromatography (30% acetone in hexanes) and obtained as a colorless solid (4.1 g, 52%).

Step 2: 2-Cyclohexyl-5-methyl-oxazole-4-carboxylic Acid Methyl Ester

(±)-2-(Cyclohexanecarbonyl-amino)-3-oxo-butyric acid methyl ester (3.9 g, 16 mmol) was added to a mixture of trifluoroacetic anhydride (15 mL) and trifluoroacetic acid (15 mL). The solution was heated to reflux for 3 hours, cooled and poured into a mixture of ice and water. The resulting mixture was neutralized with solid sodium carbonate and extracted with ethyl acetate. The combined extracts were washed with water and brine, dried over magnesium sulfate and concentrated in vacuo to an oil which solidified upon drying on high vacuum (2.4 g, 67%).

Step 3: 2-Cyclohexyl-5-methyl-oxazole-4-carboxylic Acid

2 N Sodium hydroxide (6 mL) was added to a solution of 2-cyclohexyl-5-methyl-oxazole-4-carboxylic acid methyl ester (2.4 g, 11 mmol) in methanol (20 mL). The solution was heated to reflux for 1 hour, then cooled, concentrated to about 14 of its volume and poured into 1 N hydrochloric acid. The precipitate was collected, washed with water and dried on high vacuum (1.96 g, 87%).

Step 4: 2-Cyclohexyl-5-methyl-oxazole-4-carbonyl Chloride

To a suspension of 2-cyclohexyl-5-methyl-oxazole-4-carboxylic acid (0.30 g, 1.43 mmol) in dichloromethane (5 mL) was added oxalyl chloride (0.15 mL, 1.7 mmol), followed by dimethylformamide (1 drop). After 2 hours the mixture was concentrated in vacuo to a red oil (0.28 g, 86%). ¹H NMR (400 MHz, CDCl₃) δ1.20-1.39 (m, 3H), 1.51-1.60 (m, 2H), 1.68-1.71 (m, 1H), 1.79-1.83 (m, 1H), 2.02 (dd, J=13.5, 2.7 Hz, 2H), 2.60 (s, 3H), 2.79 (m, 1H).

### Preparation 8 6,7-Dimethyl-quinoxaline-2-carboxylic Acid

Step 1: 1-(6,7-Dimethyl-quinoxalin-2-yl)-butane-(1R,2S,3R)-1,2,3,4-tetraol.

A slurry of D-glucose (3.6 g, 20 mmol) and 4,5-dimethyl-1,2-phenylenediamine (2.72 g, 20 mmol) in water (50 mL) was treated with concentrated hydrochloric acid (5 mL), acetic acid (5 mL) and hydrazine hydrate (6.6 mL). The mixture was heated to 110C in a sealed flask for 4 hours. The mixture was cooled, the solid was collected, washed with cold water and 50% ethanol/water, and dried (3.46 g, 70%).

Step 2: 6,7-Dimethyl-quinoxaline-2-carboxylic Acid

1-(6,7-Dimethyl-quinoxalin-2-yl)-butane-(1R,2S,3R)-1,2,3,4-tetraol (2.32 g, 8.3 mmol) was slurried in water and heated to reflux. Potassium permanganate (7.64 g, 49 mmol) was added in portions over 15 minutes. The reaction mixture was stirred for 10 minutes, cooled and filtered through diatomaceous earth. The filter cake was washed with 1 N sodium hydroxide and the filtrate was treated with sodium hydrogen sulfite (1 g), and acidified at 0° C. to pH 3 with concentrated hydrochloric acid. The solution was then extracted with ethyl acetate, the combined extracts were dried over sodium sulfate and concentrated to a yellow solid which was dried under high vacuum overnight (1.03 g, 62%). ¹H NMR (400 MHz, DMSO-d₆) δ7.91 (s,1H), 7.94 (s, 1H), 9.29 (s, 1H).

### Preparation 9 6.7-Dichloro-quinoxaline-2-carboxylic Acid

Prepared in an analogous manner as described in the preparation of 6,7-dimethyl-quinoxaline-2-carboxylic acid, but using 4,5-dichloro-1,2-phenylenediamine instead of 4,5-dimethyl-1,2-phenylenediamine. ¹H NMR (400 MHz, DMSO-d₆) δ 8.51 (s,1H), 8.55 (s, 1H), 9.42 (s, 1H).

### Preparation 10 6,7-Difluoro-quinoxaline-2-carboxylic Acid

Step 1: 4,5-Difluoro-1,2-phenylenediamine

4,5-Difluoro-2-nitro-aniline (10 g, 57 mmol) and 10% palladium on carbon (2.0 g, 1.9 mmol) were placed in methanol (150 mL). The mixture was hydrogenated at 50 psi in a hydrogenator. After 3 hours the reaction mixture was filtered through diatomaceous earth and concentrated to a solid which was dried overnight (8.05 g, 98%).

Steps 2 and 3: 6,7-Difluoro-quinoxaline-2-carboxylic Acid

Prepared in analogous manner to the preparation of 6,7-dimethyl-quinoxaline-2-carboxylic acid, using 4,5-difluoro-1,2-phenylenediamine instead of 4,5-dimethyl-1,2-phenylenediamine. ¹H NMR (400 MHz, DMSO-d₆) δ 8.26 (dd, J=10.8, 8.3 Hz, 1H), 8.31 (dd, J=11.0, 8.5 Hz, 1H), 9.39 (s, 1H).

### Preparation 11 2-Phenyl-oxazole-4-carboxylic Acid

2-Phenyl-oxazole-4-carboxylic acid methyl ester (Meyers and Tavares J. Org. Chem. 1996, 61, 8207) (630 mg, 3.1 mmol) was dissolved in ethanol (10 mL) and treated with 2 N sodium hydroxide. The mixture was stirred 1 hour at room temperature then acidified with 1 N hydrochloric acid and extracted with ethyl acetate. The combined extracts were washed with water and brine, dried over magnesium sulfate and concentrated. The residual white solid was dried under high vacuum (459 mg, 78%). ¹H NMR (400 MHz, CD₃OD) δ 7.26-7.32 (m, 3H), 8.10 (d, J=7.5 Hz, 2H), 8.55 (s, 1H).

### Preparation 12 2-phenyl-2H-[1,2,3]triazole-4-carboxylic Acid

Step 1: 5,6-Bis-(phenyl-hydrazono)-hexane-(1R,2S,3R)-1,2,3,4-tetraol

D-glucose (36 g, 0.2 mol), phenylhydrazine (21.6 g, 0.2 mol), water (250 mL) and acetic acid (10 mL) were combined and stirred vigorously at room temperature overnight. The precipitate was collected and washed with water. The filtrate was extracted with ethyl acetate, the combined extracts were dried over sodium sulfate and concentrated to a gum. The residue was combined with the collected precipitate and dried (9.2 g, 26%).

Step 2: 2-Phenyl-2H-[1,2,3]triazole-4-carboxylic Acid

5,6-Bis-(phenyl-hydrazono)-hexane-(1R,2S,3R)-1,2,3,4-tetraol was oxidized with potassium permanganate as described in Preparation 8, Step 2. The acid was obtained as a white solid (82%). ¹H NMR (400 MHz, DMSO-d₆) δ 7.48 (dt, J=1.5, 6.0 Hz, 1H), 7.59 (t, J=7.9 Hz, 2H), 8.03 (d, J=7.9 Hz, 2H), 8.49 (s, 1H).

### EXAMPLES

The Examples set forth hereinbelow are for illustrative purposes only. The compositions, methods, and various parameters reflected therein are intended only to exemplify various aspects and embodiments of the invention, and are not intended to limit the scope of the claimed invention in any way.

### Example 1 (S)-[5-Amino-6-oxo-6-(3,3,4,4-tetrafluoro-pyrrolidin-1-yl)-hexyl-carbamic Acid Benzyl Ester Hydrochloride

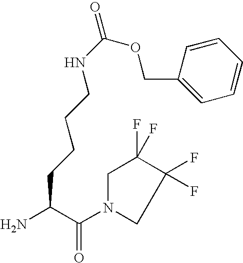

(S)-[5-Benzyloxycarbonylamino-1-(3,3,4,4-tetrafluoro-pyrrolidine-1-carbonyl)-pentyl-carbamic acid tert-butyl ester (100 mg, 0.20 mmol) was dissolved in ethyl acetate (10 mL), cooled to about 0° C. and the solution was saturated with gaseous hydrogen chloride. After about 1.5 hours at room temperature, the mixture was concentrated to dryness and the solid was dried under vacuum overnight (72 mg, 83%, melting point: 45-47° C.).

### Example 2 (S)-N-[5-Amino-6-oxo-6-(3,3,4,4-tetrafluoro-pyrrolidin-1-yl)-hexyl]-3-fluoro-benzamide Hydrochloride

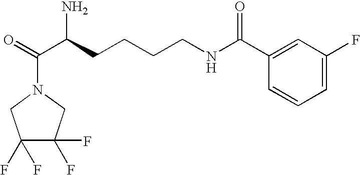

3-Fluorobenzoyl chloride (20 μL, 0.16 mmol) was added to a solution of [5-amino-1-(3,3,4,4-tetrafluoro-pyrrolidine-1-carbonyl)-pentyl]-carbamic acid tert-butyl ester (50 mg, 0.135 mmol) and triethylamine (28 μL, 0.20 mmol) in dichloromethane (2 mL) at 0° C. The reaction mixture was warmed to room temperature and after 1 hour diluted with dichloromethane, washed with 1 N sodium hydroxide, 1 N hydrochloric acid, water and brine, dried over magnesium sulfate and concentrated. The residue was purified by flash-chromatography (30% acetone in hexanes) and the product obtained as an oil. This oil was then dissolved in ether and cooled to 0° C. The solution was saturated with hydrogen chloride, warmed to room temperature, and after 1 hour, the solvent was evaporated. The residue was dried on high vacuum (35 mg, 60%). ¹H NMR (400 MHz, CD₃OD) δ 1.43-1.50 (m, 2H), 1.63-1.71 (m, 2H), 1.82-1.95 (m, 2H), 3.40 (t, J=7.1 Hz, 2H), 3.92-4.41 (m, 5H), 7.27 (dt, J=2.6 Hz, 8.2 Hz, 1H), 7.47 (m, 1H), 7.55 (td, J=2.1 Hz, 8.9 Hz, 1H), 7.63 (d, J=7.48 Hz, 1H), 8.59 (br, 1H).

Using the appropriate starting materials, Examples 3-26 were prepared in a manner analogous to that described for Example 2:

### Example 3 (S)-N-[5-Amino-6-oxo-6-(3,3,4,4-tetrafluoro-pyrrolidin-1-yl)-hexyl]-3-cyano-benzamide Hydrochloride

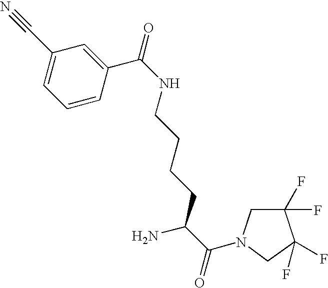

Prepared from (S)-[5-amino-1-(3,3,4,4-tetrafluoro-pyrrolidine-1-carbonyl)-pentyl]-carbamic acid tert-butyl ester and 3-cyanobenzoyl chloride. ¹H NMR (400 MHz, D₂O) δ 1.27-1.33 (m, 2H), 1.47-1.53 (m, 2H), 1.73-1.79 (m, 2H), 3.25 (t, J=6.9 Hz, 2H), 7.50 (t, J=7.9 Hz, 1H), 7.78 (dd, J=1.2 Hz, 7.9 Hz), 7.83 (m, 1H), 7.93 (s, 1H).

### Example 4 (S)-5-Methyl-2-phenyl-2H-[1,2,3]triazole-4-carboxylic Acid [5-Amino-6-(3,3-difluoro-pyrrolidin-1-yl)-6-oxo-hexyl]-amide Hydrochloride

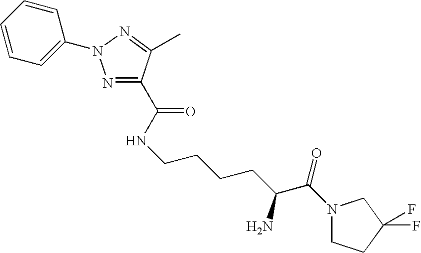

Prepared from (S)-[5-amino-1-(3,3-difluoro-pyrrolidine-1-carbonyl)-pentyl]-carbamic Acid tert-butyl ester and 5-methyl-2-phenyl-2H-[1,2,3]triazole-4-carbonyl chloride. mp 90-92° C.

### Example 5 (S)-Quinoxaline-2-carboxylic Acid [5-Amino-6-oxo-6-(3,3,4,4-tetrafluoro-pyrrolidin-1-yl)-hexyl]-amide Hydrochloride

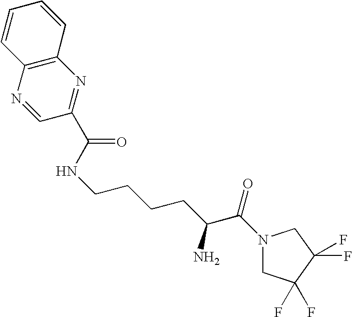

Prepared from (S)-[5-amino-1-(3,3,4,4-tetrafluoro-pyrrolidine-1-carbonyl)-pentyl]-carbamic acid tert-butyl ester and 2-quinoxaline carbonyl chloride. mp 92-95° C.

### Example 6 (S)-N-[5-Amino-6-oxo-6-(3,3,4,4-tetrafluoro-pyrrolidin-1-yl)-hexyl]-2-phenoxy-acetamide Hydrochloride

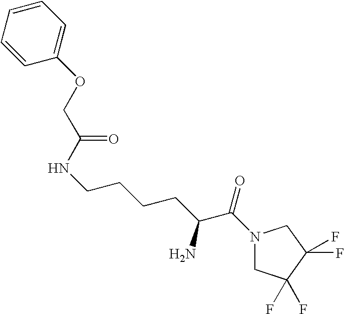

Prepared from (S)-[5-amino-1-(3,3,4,4-tetrafluoro-pyrrolidine-1-carbonyl)-pentyl]-carbamic acid tert-butyl ester and 2-phenoxyacetyl chloride. ¹H NMR (400 MHz, CD₃OD) δ1.36-1.44 (m, 2H), 1.55-1.62 (m, 2H), 1.78-1.91 (m, 2H), 3.3 (m, 2H), 3.98-4.35 (m, 5H), 6.96-7.00 (m, 3H), 7.28 (dd, J=1.2 Hz, 8.1 Hz, 2H).

### Example 7 (S)-3-(2-Chloro-phenyl-5-methyl-isoxazole-4-carboxylic Acid [5-Amino-6-oxo-6-(3,3,4,4-tetrafluoro-pyrrolidin-1-yl)-hexyl]-amide Hydrochloride

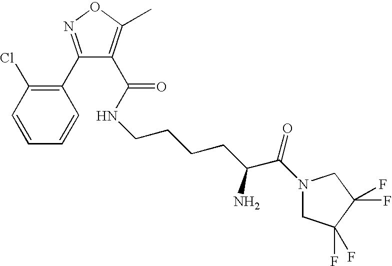

Prepared from (S)-[5-amino-1-(3,3,4,4-tetrafluoro-pyrrolidine-1-carbonyl)-pentyl]-carbamic acid tert-butyl ester and 3-(2-chloro-pheny)1-5-methyl-isoxazole-4-carbonyl chloride. ¹H NMR (400 MHz, CD₃OD) δ 1.37-1.50 (m, 2H), 1.52-1.58 (m, 2H), 1.75-1.88 (m, 2H), 2.62 (s, 3H), 3.18-3.25 (m, 2H), 3.95-4.32 (m, 5H), 7.43-7.52 (m, 4H), 7.81 (br, 1H).

### Example 8 (S)-N-[5-Amino-6-oxo-6-(3,3,4,4-tetrafluoro-pyrrolidin-1-yl)-hexyl]-2-thiophen-2-yl-acetamide Hydrochloride

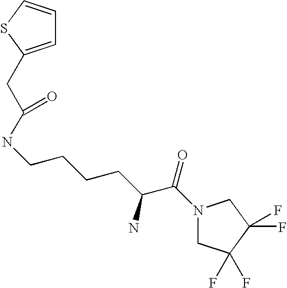

Prepared from (S)-[5-amino-1-(3,3,4,4-tetrafluoro-pyrrolidine-1-carbonyl)-pentyl]-carbamic acid tert-butyl ester and thiophene-2-acetyl chloride. ¹H NMR (400 MHz, CD₃OD) δ1.35-1.43 (m, 2H), 1.53-1.58 (m, 2H), 1.74-1.89 (m, 2H), 3.19 (t, J=7.1 Hz, 2H), 3.92-4.33 (m, 5H), 6.92-6.95 (m, 2H), 7.25 (dd, J=1.7 Hz, 4.6 Hz, 1H).

### Example 9 (S)-N-[5-Amino-6-oxo-6-(3,3,4,4-tetrafluoro-pyrrolidin-1-yl)-hexyl]-N′,N′-dimethylsulfamide Hydrochloride

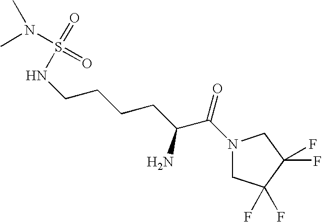

Prepared from (S)-[5-amino-1-(3,3,4,4-tetrafluoro-pyrrolidine-1-carbonyl)-pentyl]-carbamic acid tert-butyl ester and N,N-dimethyl sulfamyl chloride. ¹H NMR (400 MHz, CD₃OD) δ1.46-1.60 (m, 4H), 1.77-1.91 (m, 2H), 2.73 (s, 6H), 3.02 (t, J=6.4 Hz, 2H), 3.99-4.38 (m, 5H).

### Example 10 (S)-5-Methyl-2-phenyl-oxazole-4-carboxylic Acid [5-Amino-6-oxo-6-(3,3-4,4-tetrafluoro-pyrrolidin-1-yl)-hexyl]-amide Hydrochloride

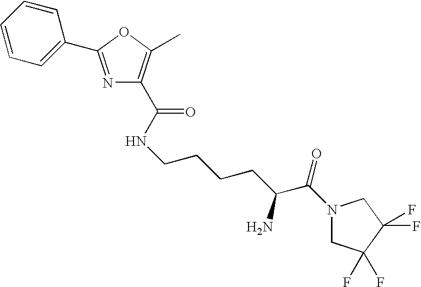

Prepared from (S)-[5-amino-1-(3,3,4,4-tetrafluoro-pyrrolidine-1-carbonyl)-pentyl]-carbamic acid tert-butyl ester and 5-methyl-2-phenyl-oxazole-4-carbonyl chloride (Cornforth et al. J., Chem. Soc. 1952, 1085). mp 111-112° C.

### Example 11 (S)-5-Methyl-2-phenyl-oxazole-4-carboxylic Acid [5-Amino-6-(3,3-difluoro-Pyrrolidin-1-yl)-6-oxo-hexyl]-amide Hydrochloride

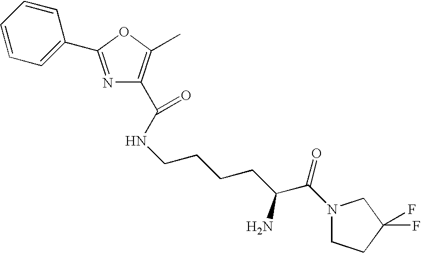

Prepared from (S)-[5-amino-1-(3,3-difluoro-pyrrolidine-1-carbonyl)-pentyl]-carbamic acid tert-butyl ester and 5-methyl-2-phenyl-oxazole4-carbonyl chloride (Cornforth et al. J., Chem. Soc. 1952, 1085). mp 109-110° C.

### Example 12 (S)-Pyrazine-2-carboxylic Acid [5-Amino-6-oxo-6-(3,3,4,4-tetrafluoro-Pyrrolidin-1-yl)-hexyl]-amide Hydrochloride

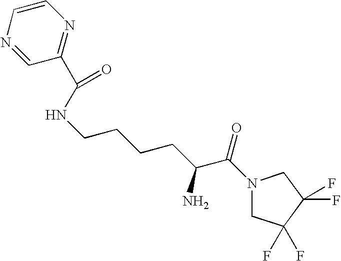

Prepared from (S)-[5-amino-1-(3,3,4,4-tetrafluoro-pyrrolidine-1-carbonyl)-pentyl]-carbamic acid tert-butyl ester and 2-pyrazine carbonyl chloride. mp 110-111° C.

### Example 13 (S)-Quinoxaline-2-carboxylic Acid [5-Amino-6-(3-fluoro-azetidin-1-yl)-6-oxo-hexyl]-amide Hydrochloride

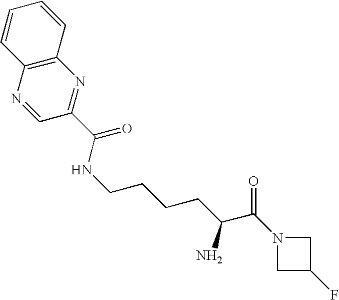

Prepared from (S)-[5-Amino-1-(3-fluoro-azetidine-1-carbonyl)-pentyl]-carbamic acid tert-butyl ester and 2-quinoxaline carbonyl chloride. mp 109-110° C.

### Example 14 (S)-5-Methyl-2-phenyl-oxazole-4-carboxylic Acid [5-Amino-6-(3-fluoro-azetidin-1-yl)-6-oxo-hexyl]-amide Hydrochloride

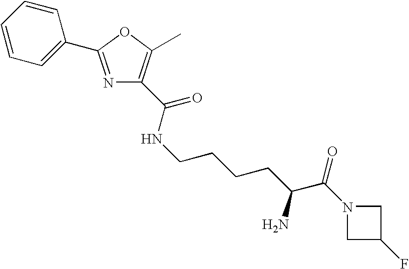

Prepared from (S)-[5-amino-1-(3-fluoro-azetidine-1-carbonyl)-pentyl]-carbamic acid tert-butyl ester and 5-methyl-2-phenyl-oxazole-4-carbonyl chloride (Cornforth et al. J., Chem. Soc. 1952, 1085). mp 75-77° C.

### Example 15 (S)-5-Methyl-2-phenyl-oxazole-4-carboxylic Acid [5-Amino-6-(3,3-difluoro-azetidin-1-yl)-6-oxo-hexyl]-amide Hydrochloride

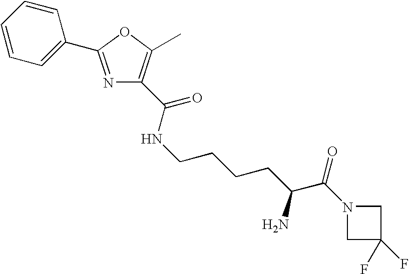

Prepared from (S)-[5-amino-1-(3,3-difluoro-azetidine-1-carbonyl)-pentyl]-carbamic acid tert-butyl ester and 5-methyl-2-phenyl-oxazole-4-carbonyl chloride (Cornforth et al. J., Chem. Soc. 1952, 1085). mp 99-101° C.

### Example 16 (S)-Quinoxaline-2-carboxylic Acid [5-Amino-6-(3,3-difluoro-azetidin-1-yl)-6-oxo-hexyl]-amide Hydrochloride

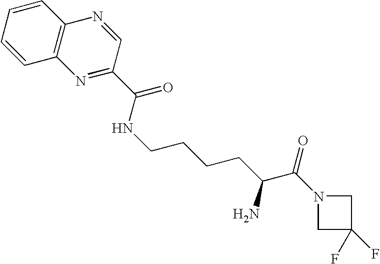

Prepared from (S)-[5-amino-1-(3,3-difluoro-azetidine-1-carbonyl)-pentyl]-carbamic acid tert-butyl ester and 2-quinoxaline carbonyl chloride. mp 109-110° C.

### Example 17 (S)-5-Methyl-2-phenyl-2H-[1,2,3]triazole-4-carboxylic Acid [5-Amino-6-(3,3-difluoro-azetidin-1-yl)-6-oxo-hexyl]-amide Hydrochloride

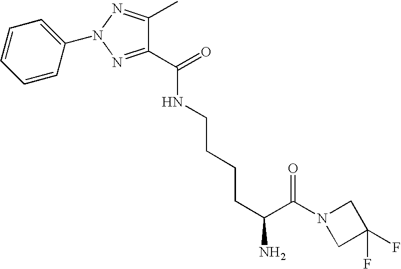

Prepared from from (S)-[5-amino-1-(3,3-difluoro-azetidine-1-carbonyl)-pentyl]-carbamic acid tert-butyl ester and 5-methyl-2-phenyl-2H-[1,2,3]triazole-4-carbonyl chloride. mp 115-117° C.

### Example 18 (S)-5-Methyl-2-phenyl-2H-[1,2,3]triazole-4-carboxylic Acid [5-Amino-6-(3-fluoro-azetidin-1-yl)-6-oxo-hexyl]-amide Hydrochloride

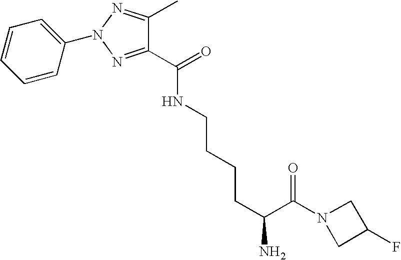

Prepared from (S)-[5-amino-1-(3-fluoro-pyrrolidine-1-carbonyl)-pentyl]-carbamic acid tert-butyl ester and 5-methyl-2-phenyl-2H-[1,2,3]triazole-4-carbonyl chloride. ¹H NMR (400 MHz, CD₃OD) δ 1.45-1.50 (m, 2H), 1.66-1.71 (m, 2H), 1.80-1.99 (m, 2H), 2.57 (s, 3H), 3.42 (t, J=5.6 Hz, 2H), 3.97-4.42 (m, 4H), 4.58-4.61 (m, 1H), 5.30 (m, 0.5H), 5.45 (m, 0.5H), 7.41 (t, J=7.1 Hz, 1H), 7.52 (t, J=7.9 Hz, 2H), 8.08 (d, J=7.9 Hz, 2H).

### Example 19 (S)-2-(4-Methoxy-phenyl)-5-methyl-oxazole-4-carboxylic Acid [5-amino-6-(3,3-difluoro-pyrrolidin-1-yl)-6-oxo-hexyl]-amide Hydrochloride

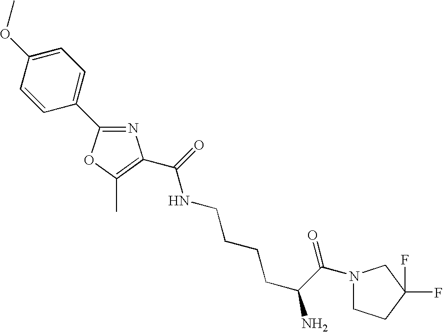

Prepared from (S)-[5-amino-1-(3,3-difluoro-pyrrolidine-1-carbonyl)-pentyl]-carbamic acid tert-butyl ester and 2-(4-methoxy-phenyl)-5-methyl-oxazole-4-carbonyl chloride. mp 113-115° C.

### Example 20 (S) 2-Phenyl-5-trifluoromethyl-oxazole-4-carboxylic Acid [5-Amino-6-(3,3-difluoro-pyrrolidin-1-yl)-6-oxo-hexyl]-amide Hydrochloride

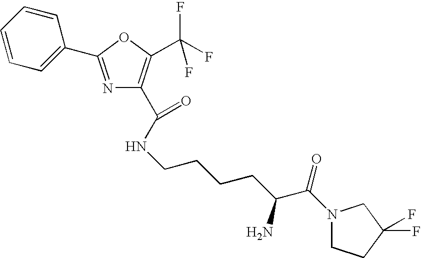

Prepared from (S)-[5-amino-1-(3,3-difluoro-pyrrolidine-1-carbonyl)-pentyl]-carbamic acid tert-butyl ester and 2-phenyl-5-trifluoromethyl-oxazole-4-carboxylic acid chloride (made from 2-phenyl-5-trifluoromethyl-oxazole-4-carboxylic acid and oxalyl chloride). ¹H NMR (400 MHz, CD₃OD) δ 1.46-1.53 (m, 2H), 1.66-1.70 (m, 2H), 1.81-1.96 (m, 2H), 2.39-2.56 (m, 4H), 3.42 (m, 2H), 3.66-4.05 (m, 4H), 4.13 (dd, J=4.8 Hz, 7.7 Hz, 0.5H), 4.21 (dd, J=5.0 Hz, 7.9 Hz, 0.5H), 7.55-7.64 (m, 3H), 8.12 (d, J=7.7 Hz, 1H).

### Example 21 (S)-N-[5-Amino-(3,3-difluoro-pyrrolidin-1-yl)-6-oxo-6-hexyl]-2-phenoxy-acetamide Hydrochloride

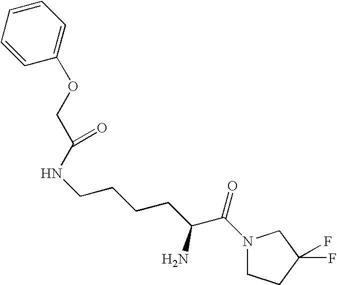

Prepared from (S)-[5-amino-1-(3,3-difluoro-pyrrolidine-1-carbonyl)-pentyl]-carbamic acid tert-butyl ester and 2-phenoxyacetyl chloride. ¹H NMR (400 MHz, CD₃OD) δ 1.38-1.43 (m, 2H), 1.55-1.62 (m, 2H), 1.79-1.90 (m, 2H), 2.40-2.56 (m, 2H), 3,3 (m, 2H), 3.64-4.19 (m, 5H), 4.49 (s, 2H), 6.96-7.00 (m, 3H), 7.27-7.32 (m, 2H).

### Example 22 (2RS)-2,3-Dihydro-benzofuran-2-carboxylic Acid [(5S)-5-amino-6-(3,3-difluoro-pyrrolidin-1-yl)-6-oxo-hexyl]-amide Hydrochloride

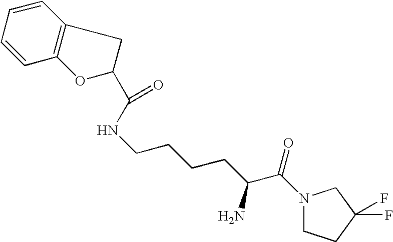

Prepared from (S)-[5-amino-1-(3,3-difluoro-pyrrolidine-1-carbonyl)-pentyl]-carbamic acid tert-butyl ester and 2,3-dihydro-benzofuran-2-carbonyl chloride (made from 2,3-dihydro-benzofuran-2-carboxylic acid, Stoermer and Koenig, Chem. Ber. 1906, 39, 496). mp 139-140° C.

### Example 23 (S)-N-[5-Amino-6-(3,3-difluoro-pyrrolidin-1-yl)-6-oxo-hexyl]-2-(4-fluoro-phenoxy)-acetamide Hydrochloride

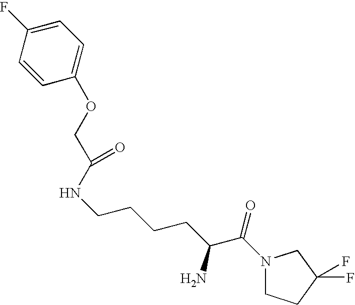

Prepared from (S)-[5-amino-1-(3,3-difluoro-pyrrolidine-1-carbonyl)-pentyl]-carbamic acid tert-butyl ester and (4-fluoro-phenoxy)-acetyl chloride (made from (4-fluoro-phenoxy)-acetic acid by treatment with oxalyl chloride and a catalytic amount of dimethylformamide in dichloromethane). ¹H NMR (400 MHz, CD₃OD) δ 1.38-1.45 (m, 2H), 1.55-1.60 (m, 2H), 1.80-1.88 (m, 2H), 2.40-2.53 (m, 2H), 3.3 (m, 2H), 3.65-4.21 (m, 5H), 4.46 (s, 2H), 6.95-7.05 (m, 4H).

### Example 24 (S)-3-Methyl-quinoxaline-2-carboxylic Acid [5-amino-6-oxo-6-(3,3,4,4-tetrafluoro-pyrrolidin-1-yl)-hexyl]-amide Hydrochloride

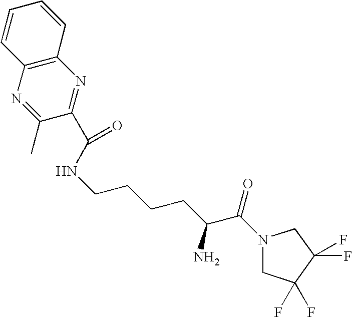

Prepared from (S)-[5-amino-1-(3,3,4,4-tetrafluoro-pyrrolidine-1-carbonyl)-pentyl]-carbamic acid tert-butyl ester and 3-methyl-2-quinoxaline carbonyl chloride. mp 107-109° C.

### Example 25 (S)-3-Methylquinoxaline-2-carboxylic Acid [5-Amino-6-(3-fluoro-azetidin-1-yl)-6-oxo-hexyl]-amide Hydrochloride

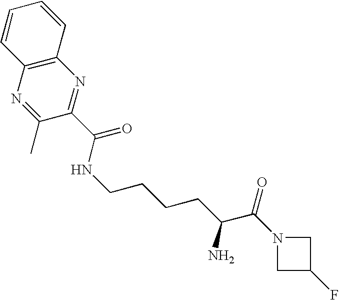

Prepared from (S)-[5-amino-1-(3-fluoro-azetidine-1-carbonyl)-pentyl]-carbamic acid tert-butyl ester and 3-methyl-2-quinoxaline carbonyl chloride. mp 99-100° C.

### Example 26 (S)-3-Methyl-quinoxaline-2-carboxylic Acid [5-Amino-6-(3,3-difluoro-azetidin-1-yl)-6-oxo-hexyl]-amide Hydrochloride

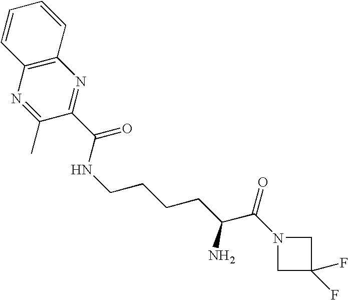

Prepared from (S)-[5-amino-1-(3,3-difluoro-azetidine-1-carbonyl)-pentyl]-carbamic acid tert-butyl ester and 3-methyl-2-quinoxaline carbonyl chloride. mp 97-99° C.

### Example 27 (S)-5-Methyl-2-phenyl-2H-[1,2,3]triazole-4-carboxylic Acid [5-Amino-6-oxo-6-(3,3,4,4-tetrafluoro-pyrrolidin-1-yl)-hexyl]-amide Hydrochloride

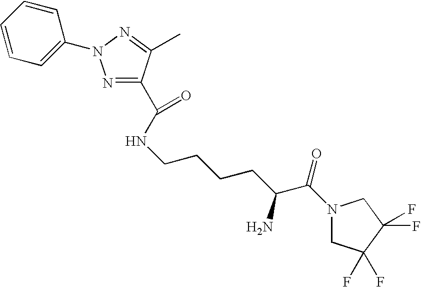

1-(3-Dimethylaminopropyl)-3-ethylcarbodiimide methiodide (0.24 g, 0.54 mmol) was added to a solution of (S)-[5-amino-1-(3,3,4,4-tetrafluoro-pyrrolidine-1-carbonyl)-pentyl]-carbamic acid tert-butyl ester (0.20 g, 0.54 mmol) and 4-dimethylaminopyridine (0.20 g, 1.6 mmol) in DMF (5 mL). The mixture was stirred at room temperature overnight, diluted with water and extracted with ethyl acetate. The combined extracts were washed with 1 N sodium hydroxide, 1 N hydrochloric acid, water and brine, dried over magnesium sulfate and concentrated to dryness. The product was purified by flash-chromatography (30% acetone in hexanes) and obtained as an oil (15 mg). The oil was dissolved in ether, cooled to 0° C., and the solution was saturated with hydrogen chloride, warmed to room temperature and stirred for 30 minutes, then concentrated in vacuo to a glassy solid. ¹H NMR (400 MHz, CD₃OD) δ 1.47-1.53 (m, 2H), 1.66-1.71 (m, 2H), 1.83-1.97 (m, 2H), 2.57(s,3H), 3.42 (m, 2H), 3.97-4.38 (m, 5H), 7.41 (m, 1H), 7.52 (m, 2H), 8.07 (m, 2H).

### Example 28 (S)-6,7-dimethylquinoxaline-2-carboxylic Acid [5-Amino-6-(3-fluoro-azetidin-1-yl)-6-oxo-hexyl]-amide Hydrochloride

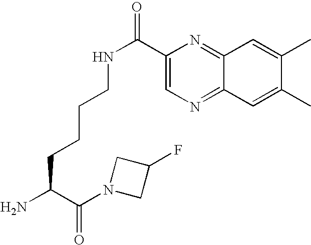

1-(3-Dimethylaminopropyl)-3-ethylcarbodiimide methiodide (0.24 g, 0.54 mmol) was added to a solution of (S)-[5-amino-1-(3-fluoro-azetidine-1-carbonyl)-pentyl]-carbamic acid tert-butyl ester (100 mg, 0.33 mmol), 6,7-dimethyl-2-quinoxaline carboxylic acid (66 mg, 0.33 mmol) and hydroxybenzotriazole (58 mg, 0.43 mmol) in dichloromethane (2.5 mL). The mixture was stirred overnight at room temperature, then concentrated and the residue was partitioned between ethyl acetate and 1 N sodium hydroxide. The organic layer was washed with 1 N sodium hydroxide, 1 N hydrochloric acid and brine, dried over sodium sulfate and concentrated. The residue was purified by flash-chromatography (hexanes/acetone, 3:1, then 2:1 and 1:1) and the product was obtained as a white foam (103 mg, 64%).

The product was dissolved in ethyl acetate (2 mL), the solution was cooled to 0° C., saturated with hydrogen chloride, and stirred for 1 hour at room temperature. Ether (1 mL) was added and after precipitation the liquid was decanted. The rest of the solvent was evaporated and the white solid was dried under vacuum (85 mg, 95%). ¹H NMR (400 MHz, CD₃OD) δ 1.47-1.52 (m, 2H), 1.71-1.80 (m, 2H), 1.82-1.95 (m, 2H), 2.55 (s, 6H), 3.48-3.56 (m, 2H), 3.98-4.15 (m, 2H), 4.00-4.43 (m, 4H), 4.58-4.64 (m, 1H), 5.31 (m, 0.5H), 5.46 (m, 0.5H), 7.91 (s, 1H), 7.98 (s, 1H), 9.38 (s, 1H).

Using the appropriate starting materials, Examples 29-40 were prepared in a manner analogous to that described for Example 28:

### Example 29 (S)-6,7-Dimethylquinoxaline-2-carboxylic Acid [5-Amino-6-(3,3-difluoro-azetidin-1-yl)-6-oxo-hexyl]-amide Hydrochloride

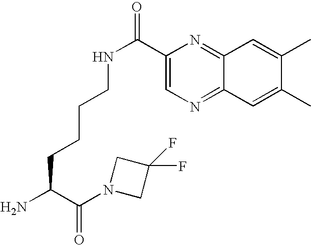

Prepared from (S)-[5-amino-1-(3,3-difluoro-azetidine-1-carbonyl)-pentyl]-carbamic acid tert-butyl ester and 6,7-dimethyl-2-quinoxaline carboxylic acid. ¹H NMR (400 MHz, CD₃OD) δ 1.44-1.52 (m, 2H), 1.71-1.80 (m, 2H), 1.82-1.95 (m, 2H), 2.55 (s, 6H), 3.52 (m, 2H), 4.07 (m, 1H), 4.28-4.50 (m, 2H), 4.58-4.64 (m, 1H), 4.72 (t, J=11.8 Hz, 2H), 7.91 (s, 1H), 7.98 (s, 1H), 9.38 (s, 1H).

### Example 30 (S)-6,7-Dichloro-quinoxaline-2-carboxylic Acid [5-Amino-6-(3-fluoro-azetidin-1-yl)-6-oxo-hexyl]-amide Hydrochloride

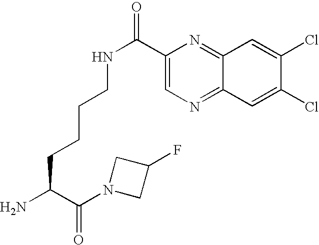

Prepared from (S)-[5-amino-1-(3-fluoro-azetidine-1-carbonyl)-pentyl]-carbamic acid tert-butyl ester and 6,7-dichloro-2-quinoxaline carboxylic acid. ¹H NMR (400 MHz, CD₃OD) δ 1.44-1.54 (m, 2H), 1.69-1.78 (m, 2H), 1.81-1.92 (m, 2H), 3.49-3.55 (m, 2H), 3.97-4.17 (m, 2H), 4.24-4.43 (m, 2H), 4.56-4.67 (m, 1H), 5.33 (m, 0.5H), 5.46 (m, 0.5H), 8.38 (s, 1H), 8.42 (s, 1H), 9.51 (s, 1H).

### Example 31 (S)-6,7-Dichloro-quinoxaline-2-carboxylic Acid [5-Amino-6-(3,3-difluoro-azetidin-1-yl)-6-oxo-hexyl]-amide Hydrochloride

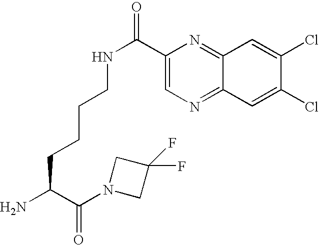

Prepared from (S)-[5-amino-1-(3,3-difluoro-azetidine-1-carbonyl)-pentyl]-carbamic acid tert-butyl ester and 6,7-dichloro-2-quinoxaline carboxylic acid. ¹H NMR (400 MHz, CD₃OD) δ 1.46-1.53 (m, 2H), 1.70-1.77 (m, 2H), 1.82-1.98 (m, 2H), 3.52 (m, 2H), 4.06 (t, J=6.2 Hz, 1H), 4.34 (q, J=12.0 Hz, 1H), 4.44 (q, J=12.5 Hz, 1H), 4.72 (t, J=11.8 Hz, 2H), 8.38 (s, 1H), 8.42 (s, 1H), 9.51 (s, 1H).

### Example 32 (S)-6,7-Difluoro-quinoxaline-2-carboxylic Acid [5-Amino-6-(3-fluoro-azetidin-1-yl)-6-oxo-hexyl]-amide Hydrochloride

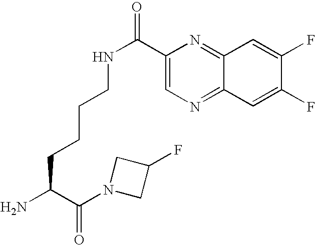

Prepared from (S)-[5-amino-1-(3-fluoro-azetidine-1-carbonyl)-pentyl]-carbamic acid tert-butyl ester and 6,7-difluoro-2-quinoxaline carboxylic acid. ¹H NMR (400 MHz, CD₃OD) δ 1.45-1.54 (m, 2H), 1.69-1.78 (m, 2H), 1.81-1.92 (m, 2H), 3.51-3.56 (m, 2H), 3.96-4.16 (m, 2H), 4.24-4.43 (m, 2H), 4.58-4.67 (m, 1H), 5.39 (br d, J=58 Hz, 1H), 8.02-8.10 (m, 2H), 9.49 (s, 1H).

### Example 33 (S)-6,7-Dichloro-quinoxaline-2-carboxylic Acid [5-Amino-6-(3,3-difluoro-azetidin-1-yl)-6-oxo-hexyl]-amide Hydrochloride

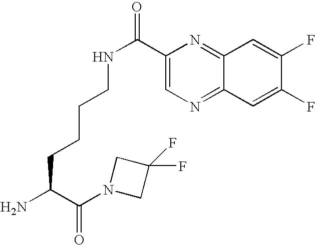

Prepared from (S)-[5-amino-1-(3,3-difluoro-azetidine-1-carbonyl)-pentyl]-carbamic acid tert-butyl ester and 6,7-difluoro-2-quinoxaline carboxylic acid. ¹H NMR (400 MHz, CD₃OD) δ 1.46-1.53 (m, 2H), 1.70-1.77 (m, 2H), 1.82-1.98 (m, 2H), 3.52 (m, 2H), 4.06 (t, J=6.4 Hz, 1H), 4.33 (q, J=12.0 Hz, 1H), 4.45(q, J=12.0 Hz, 1H), 4.72 (t, J=11.8 Hz, 2H), 8.02-8.10 (m, 2H), 9.49 (s, 1H).

### Example 34 (S)-2-Phenyl-oxazole-4-carboxylic Acid [5-Amino-6-(3,3-difluoro-azetidin-1-yl)-6-oxo-hexyl]-amide Hydrochloride

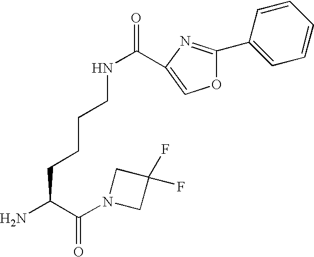

Prepared from (S)-[5-amino-1-(3,3-difluoro-azetidine-1-carbonyl)-pentyl]-carbamic acid tert-butyl ester and 2-phenyl-oxazole-4-carboxylic acid. ¹H NMR (400 MHz, CD₃OD) δ 1.44-1.51 (m, 2H), 1.65-1.72 (m, 2H), 1.79-1.96 (m, 2H), 3.43 (m, 2H), 4.05 (t, J=6.4 Hz, 1H), 4.36 (q, J=12.6 Hz, 1H), 4.45 (q, J=12.6 Hz, 1H), 4.72 (t, J=11.8 Hz, 2H), 7.49-7.56 (m, 3H), 8.06-8.09 (m, 2H), 8.41 (s, 1H).

### Example 35 (S)-2-Phenyl-oxazole-4-carboxylic Acid [5-Amino-6-(3,3-difluoro-pyrrolidin-1-yl)-6-oxo-hexyl]-amide Hydrochloride

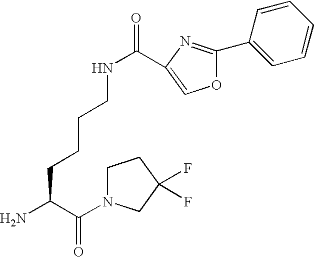

Prepared from (S)-[5-amino-1-(3,3-difluoro-pyrrolidine-1-carbonyl)-pentyl]-carbamic acid tert-butyl ester and 2-phenyl-oxazole-4-carboxylic acid. ¹H NMR (400 MHz, CD₃OD) δ 1.46-1.51 (m, 2H), 1.66-1.70 (m, 2H), 1.86-1.92 (m, 2H), 2.39-2.60 (m, 2H), 3.43 (m, 2H), 3.63-3.89 (m, 4H), 4.19 (ddd, J=32.4, 7.3, 5.2 Hz, 1H), 7.50-7.54 (m, 3H), 8.07-8.09 (m, 2H), 8.41 (s, 1H).

### Example 36 (S)-2-Phenyl-2-H-[1,2,3]triazole-4-carboxylic Acid [5-Amino-6-(3,3-difluoro-pyrrolidin-1-yl)-6-oxo-hexyl]-amide Hydrochloride

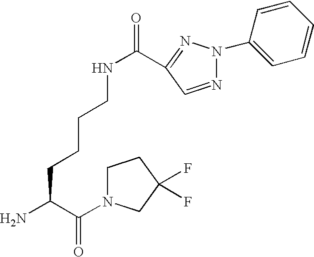

Prepared from (S)-[5-amino-1-(3,3-difluoro-pyrrolidine-1-carbonyl)-pentyl]-carbamic acid tert-butyl ester and 2-phenyl-2H-[1,2,3]triazole-4-carboxylic acid.

¹H NMR (400 MHz, CD₃OD) δ 1.47-1.53 (m, 2H), 1.67-1.70 (m, 2H), 1.85-1.95 (m, 2H), 2.38-2.57 (m, 2H), 3.44 (m, 2H), 3.64-4.06 (m, 4H), 4.19 (td, J=6.1, 32.9 Hz, 1H), 7.45 (t, J=7.5 Hz, 1H), 7.55 (t, J=7.9 Hz, 2H), 8.12 (d, J=7.9 Hz, 2H), 8.24 (s, 1H).

### Example 37 (S)-2-(4-Chloro-phenyl)-5-methyl-2-H-[1,2,3]triazole-4-carboxylic Acid [5-Amino-6-(3,3-difluoro-pyrrolidin-1-yl)-6-oxo-hexyl]-amide Hydrochloride

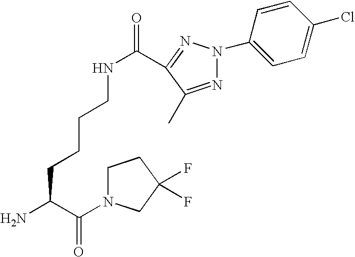

Prepared from (S)-[5-amino-1-(3,3-difluoro-pyrrolidine-1-carbonyl)-pentyl]-carbamic acid tert-butyl ester and 2-(4-chloro-phenyl)-5-methyl-2H-[1,2,3]triazole-4-carboxylic acid. ¹H NMR (400 MHz, CD₃OD) δ 1.45-1.53 (m, 2H), 1.65-1.70 (m, 2H), 1.80-1.95 (m, 2H), 2.38-2.57 (m, 2H), 2.57 (s, 3H), 3.41 (t, J=6.0 Hz, 2H), 3.60-4.10 (m, 4H), 4.20 (td, J=6.1, 33 Hz, 1H), 7.53 (dd, J=2.1, 7.1 Hz, 2H), 8.06 (dd, J=1.9, 6.9 Hz, 2H), 8.55 (br, 1H).

### Example 38 (S)-2-Phenyl-2H-[1,2,3]triazole-4-carboxylic Acid [5-Amino-6-(3-fluoro-azetidin-1-yl)-6-oxo-hexyl]-amide Hydrochloride

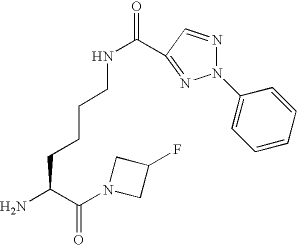

Prepared from (S)-[5-amino-1-(3-fluoro-azetidine-1-carbonyl)-pentyl]-carbamic acid tert-butyl ester and 2-phenyl-2H-[1,2,3]triazole-4-carboxylic acid. ¹H NMR (400 MHz, CD₃OD) δ 1.45-1.52 (m, 2H), 1.66-1.74 (m, 2H), 1.80-1.93 (m, 2H), 3.42-3.53 (m, 2H), 3.98-4.17 (m, 2H), 4.23-4.42 (m, 2H), 4.56-4.66 (m, 1H), 5.32 (m, 0.5H), 5.46 (m, 0.5H), 7.45 (t, J=7.5 Hz, 1H), 7.55 (t, J=7.9 Hz, 2H), 8.12 (d, J=7.9 Hz, 1H), 8.25 (d, J=4.1 Hz, 1H), 8.72 (br, 1H).

### Example 39 (S)-2-Phenyl-2H-[1,2,3]triazole-4-carboxylic Acid [5-Amino-6-(3,3-difluoro-azetidin-1-yl)-6-oxo-hexyl]-amide Hydrochloride

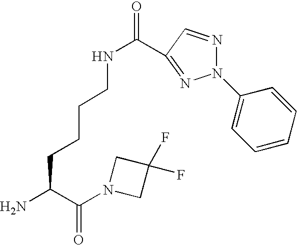

Prepared from (S)-[5-amino-1-(3,3-difluoro-azetidine-1-carbonyl)-pentyl]-carbamic acid tert-butyl ester and 2-phenyl-2H-[1,2,3]triazole-4-carboxylic acid. ¹H NMR (400 MHz, CD₃OD) δ 1.44-1.52 (m, 2H), 1.65-1.73 (m, 2H), 1.80-1.97 (m, 2H), 3.45 (m, 2H), 4.06 (t, J=6.4 Hz, 1H), 4.35 (q, J=12.0 Hz, 1H), 4.45 (q, J=12.3 Hz, 1H), 4.72 (t, J=11.6 Hz, 2H), 7.45 (t, J=7.3 Hz, 1H), 7.55 (t, J=7.9 Hz, 2H), 8.12 (d, J=7.5 Hz, 1H), 8.25 (s, 1H), 8.73 (br, 1H).

### Example 40 (S)-5-Methyl-1-phenyl-1-H-pyrazole-4-carboxylic Acid [5-Amino-6-(3,3-difluoro-pyrrolidin-1-yl)-6-oxo-hexyl]-amide Hydrochloride

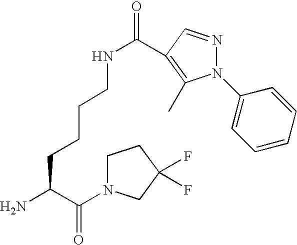

Prepared from (S)-[5-amino-1-(3,3-difluoro-pyrrolidine-1-carbonyl)-pentyl]-carbamic acid tert-butyl ester and 5-methyl-1-phenyl-1H-pyrazole-4-carboxylic acid. ¹H NMR (400 MHz, CD₃OD) δ 1.45-1.53 (m, 2H), 1.60-1.70 (m, 2H), 1.80-1.95 (m, 2H), 2.35-2.50 (m, 2H), 2.50 (s, 3H), 3,36 (m, 2H), 3.61-4.15 (m, 4H), 4.20 (td, J=6.1, 33 Hz, 1H), 7.33 (d, J=7.1 Hz, 2H), 7.51-7.58 (m, 3H), 8.00 (s, 1H).

### Example 41 (S)-2-Phenyl-oxazole-4-carboxylic Acid [5-Amino-6-(3-fluoro-azetidin-1-yl)-6-oxo-hexyl]-amide Trifluoroacetate

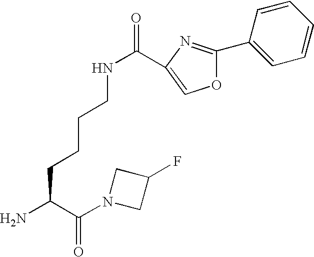

The coupling reaction between (S)-[5-amino-1-(3-fluoro-azetidine-1-carbonyl)-pentyl]-carbamic acid tert-butyl ester and 2-phenyl-oxazole-4-carboxylic acid was performed as described in Example 29. The intermediate (S)-{1-(3-fluoro-azetidine-1-carbonyl)-5-[(2-phenyl-oxazole-4-carbonyl)-amino]-pentyl}-carbamic Acid tert-butyl ester (116 mg, 0.24 mmol) was dissolved in dichloromethane (1 mL) and treated with trifluoroacetic aid (1 mL). After 20 minutes the solvent was evaporated to dryness. ¹H NMR (400 MHz, CD₃OD) δ 1.43-1.51 (m, 2H), 1.63-1.72 (m, 2H), 1.78-1.86 (m, 2H), 3.43 (m, 2H), 3.97-4.16 (m, 2H), 4.24-4.42 (m, 2H), 4.56-4.65 (m, 1H), 5.39 (br d, J=57 Hz, 1H), 8.06-8.09 (m, 2H), 8.42 (s, 1H).

### Example 42 (S)-1-[5-Amino-6-oxo-6-(3,3,4,4-tetrafluoro-pyrrolidin-1-yl)-hexyl]-3-benzyl-urea

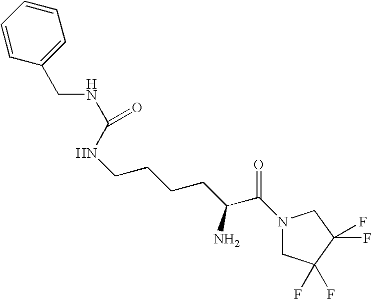

Benzyl isocyanate (33 μL, 0.27 mmol) was added to a solution of (S)-[5-amino-1-(3,3,4,4-tetrafluoro-pyrrolidine-1-carbonyl)-pentyl]-carbamic acid tert-butyl ester (0.10 g, 0.27 mmol) and N-methylmorpholine (38 μL, 0.35 mmol) in dichloromethane (2 mL). The mixture was stirred overnight, diluted with dichloromethane, washed with 1 N sodium hydroxide, 1 N hydrochloric acid, water and brine, dried over magnesium sulfate and concentrated. The product was purified by flash-chromatography (40% acetone/hexanes) and obtained pure as a colorless foam. This foam was dissolved in ethyl acetate (2 mL) and the solution was cooled to 0° C. and saturated with hydrogen chloride. After 1 hour at room temperature the solvent was evaporated and the resulting solid was dried on high vacuum (85 mg, 71%). mp 89-92° C.

### Example 43 (S)-4-Benzyl-piperidine-1-carboxylic Acid [5-Amino-6-oxo-6-(3,3,4,4-tetrafluoro-pyrrolidin-1-yl)-hexyl]-amide

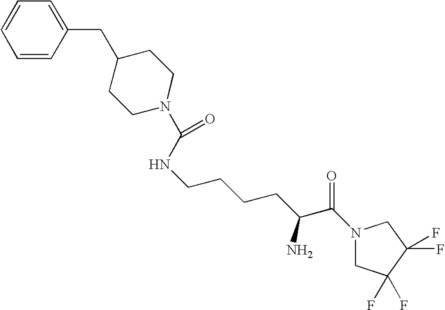

To a vigorously stirred mixture of (S)-[5-amino-1-(3,3,4,4-tetrafluoro-pyrrolidine-1-carbonyl)-pentyl]-carbamic acid tert-butyl ester (0.20 g, 0.54 mmol) in dichloromethane (3 mL) and saturated sodium bicarbonate (3 mL), cooled to 0° C., was added triphosgene (53 μg, 0.18 mmol). After 1 hour at 0° C. the mixture was diluted with chloroform and the layers were separated. The organic phase was dried over magnesium sulfate and concentrated to yield (S)-[5-isocyanato-1-(3,3,4,4-tetrafluoro-pyrrolidine-1-carbonyl)-pentyl]-carbamic acid tert-butyl ester as a colorless oil (0.18 g, 85%).

The isocyanate was reacted with 4-benzylpiperidine and the product was treated with hydrogen chloride, as described for Example 43. mp 68-70° C.

1. A compound of formula Ia, 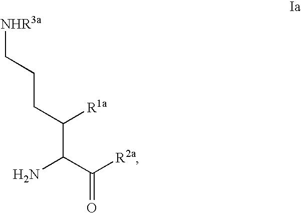
a prodrug or stereoisomer thereof, or a pharmaceutically acceptable salt of the compound, prodrug, or stereoisomer, wherein:
R^{1a} is hydrogen, (C₁-C₈)alkyl, or (C₃-C₈)cycloalkyl;
R^{2a} is 3-fluoroazetidin-1-yl; 3,3-difluoroazetidin-1-yl; 3,4-difluoropyrrolidin-1-yl; 3,3,4-trifluoropyrrolidin-1-yl; 3,3,4,4-tetrafluoropyrrolidin-1-yl, 3-fluoropiperidin-1-yl; 4-fluoropiperidin-1-yl; 3,4-difluoropiperidin-1-yl; 3,3-difluoropiperidin-1-yl; 3,5-difluoropiperidin-1-yl; 3,4,5-trifluoropiperidin-1-yl; 3,3,4-trifluoropiperidin-1-yl; 3,3,5-trifluoropiperidin-1-yl; 3,4,4-trifluoropiperidin-1-yl; 3,3,4,4-tetrafluoropiperidin-1-yl; 3,3,4,5-tetrafluoropiperidin-1-yl; 3,3,5,5-tetrafluoropiperidin-1-yl; 3,3,4,4,5-pentafluoropiperidin-1-yl; 3,3,4,5,5-pentafluoropiperidin-1-yl; or 3,3,4,4,5,5-hexafluoropiperidin-1-yl;
R^{3a} is —COR^{4a}, —COOR^{5a}, —CONR^{6a}R^{7a}, or —SO₂NR^{6a}R^{7a};
R^{4a} and R^{5a} are (A) (C₁-C₈)alkyl; (B) (C₃-C₈)cycloalkyl; (C) phenyl(C₀-C₈)alkyl; (D) phenoxy(C₁-C₈)alkyl; (E) a five- or six-membered unsaturated, partially saturated or saturated heterocyclyl(C₀-C₈)alkyl, said heterocyclyl comprising 1 to 3 of N, O, or S;
wherein said phenyl, phenoxy, and heterocyclyl are optionally and independently substituted with 1 to 3 of: (C₁-C₈)alkyl; (C₃-C₈)cycloalkyl; cyano; halo; (C₁-C₈)alkylsulfonyl; (C₁-C₈)alkylsulfonyloxy; phenyl(C₁-C₈)alkoxy; or phenyl optionally substituted with 1 to 3 of: (C₁-C₈)alkyl; halo; (C₁-C₈)alkoxy; cyano; hydroxy; trifluoromethyl; (C₁-C₈)alkylsulfonyl; (C₁-C₈)alkylsulfonyloxy; or phenyl(C₁-C₈)alkoxy; or,
(F) a nine- or ten-membered fused heterocyclyl, said fused heterocyclyl comprising 1 to 5 of: N, O, or S, and said fused heterocyclyl is optionally substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl, optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy;
R^{6a} and R^{7a} are taken separately and independently (A) hydrogen; (B) (C₁-C₈)alkyl; (C) (C₃-C₈)cycloalkyl; (D) phenyl(C₁-C₈)alkyl; (E) phenoxy(C₁-C₈)alkyl; or (F) a five- or six-membered unsaturated, partially saturated, or saturated heterocycl(C₀-C₈)alkyl, said heterocyclyl comprising 1 to 3 of N, O, or S;
wherein said phenyl, phenoxy, and heterocyclyl are optionally and independently substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy; or
R^{6a} and R^{7a} are taken together to form a 4- to 8-membered ring, whch is optionally substituted with (C₁-C₈)alkyl, (C₃-C₈)cycloalkyl, (C₁-C₈)alkoxy, phenyl, (C₁-C₈)alkoxy, or phenyl(C₁-C₈)alkyl.

2. A compound of claim 1 wherein R^{1a} is hydrogen.

3. A compound of claim 1 wherein R^{2a} is 3,4-difluoropyrrolidin-1-yl or 3,3,4,4-tetrafluoropyrrolidin-1-yl.

4. A compound of claim 1 wherein R^{2a} is 3-fluoroazetidin-1-yl or 3-3-difluoroazetidin-1-yl.

5. A compound of claim 1 wherein R^{3a} is COR^{4a}.

6. A compound of claim 5 where R^{4a} is:
(I) a five- or six-membered unsaturated, partially saturated or saturated heterocycl(C₀-C₈)alkyl, wherein said heterocycl comprises 1 to 3 of N, O, or S; and wherein said heterocycl is optionally substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl, optionally substituted with 1 to 3 of: (a) (C₁-C₈)alkyl; (b) halo; (c) (C₁-C₈)alkoxy; (d) cyano; (e) hydroxy; (f) trifluoromethyl; (g) (C₁-C₈)alkylsulfonyl; (h) (C₁-C₈)alkylsulfonyloxy; or (i) phenyl(C₁-C₈)alkoxy; or
(II) a nine- or ten-membered fused heterocycl, wherein said fused heterocyclyl comprises 1 to 5 of: N, O; or S, and wherein said fused heterocycl is optionally substituted with 1 to 3 of: (i) (C₁-C₈)alkyl; (ii) (C₃-C₈)cycloalkyl; (iii) cyano; (iv) halo; (v) (C₁-C₈)alkylsulfonyl; (vi) (C₁-C₈)alkylsulfonyloxy; (vii) phenyl(C₁-C₈)alkoxy; or (viii) phenyl, optionally substituted with 1 to 3 of: (a) (C₁-C₈)alkyl; (b) halo; (c) (C₁-C₈)alkoxy; (d) cyano; (e) hydroxy; (f) trifluoromethyl; (9) (C₁-C₈)alkylsulfonyl; (h) (C₁-C₈)alkylsulfonyloxy; or (i) phenyl(C₁-C₈)alkoxy.

7. A compound of claim 1 having an S configuration at the stereogenic carbon atom adjacent to the primary amine.

8. A pharmaceutical composition comprising a compound of claim 1 or 7, a prodrug or stereoisomer thereof, or a pharmaceutically acceptable salt of the compound, prodrug, or stereoisomer, and a pharmaceutically acceptable vehicle, carrier, or diluent.

9. A pharmaceutical composition comprising:
a) a compound of claim 1 or 7, a prodrug or stereoisomer thereof, or a pharmaceutically acceptable salt of the compound, prodrug, or stereoisomer;
b) a compound selected from insulin or an insulin analog; insulinotropin; a biguanide; an α₂-antagonist; an imidazoline; a glitazone; an aldose reductase inhibitor; a glycogen phosphorylase inhibitor; a sorbitol dehydrogenase inhibitor; a fatty acid oxidation inhibitor; an α-glucosidase inhibitor; a β-agonist; phosphodiesterase inhibitor; a lipid-lowering agent; an antiobesity agent; vanadate; a vanadium complex; a peroxovanadium complex; an amylin antagonist; a glucagon antagonist; a growth hormone secretagogue; a gluconeogenesis inhibitor; a somatostatin analog; an inhibitor of renal glucose; an antilipolytic agent; or a prodrug of said compound or a pharmaceutically acceptable salt of said compound or prodrug; and,
c) a pharmaceutically acceptable carrier, vehicle, or diluent.

10. A method of inhibiting dipeptidyl peptidase-IV in a mammal comprising administering to said mammal in need of such treatment a therapeutically effective amount of a compound of claim 1 or 7, a prodrug or stereoisomer thereof, or a pharmaceutically acceptable salt of the compound, prodrug, or stereoisomer, and a pharmaceutically acceptable vehicle, carrier, or diluent.

11. A method of treating a condition mediated by dipeptidyl peptidase-IV in a mammal comprising administering to said mammal in need of such treatment a therapeutically effective amount of a compound of claim 1 or 7, a prodrug or stereoisomer thereof, or a pharmaceutically acceptable salt of the compound, prodrug, or stereoisomer, and a pharmaceutically acceptable vehicle, carrier, or diluent.

12. The method of claim 11 wherein the condition is type 2 diabetes, metabolic syndrome, hyperglycemia, impaired glucose tolerance, glucosuria, metabolic acidosis, arthritis, cataracts, diabetic neuropathy, diabetic nephropathy, diabetic retinopathy, diabetic cardiomyopathy, type 1 diabetes, obesity, a diabetic condition exacerbated by obesity, hypertension, hyperlipidemia, atherosclerosis, osteoporosis, osteopenia, frailty, bone loss, bone fracture, acute coronary syndrome, infertility due to polycystic ovary syndrome, disease progression in type 2 diabetes, anxiety, depression, insomnia, chronic fatigue, epilepsy, an eating disorder, chronic pain, alcohol addiction, a disease associated with intestinal motility, an ulcer, irritable bowel syndrome, inflammatory bowel syndrome or short bowel syndrome.

13. The method of claim 12 wherein the condition is type 2 diabetes.

14. A compound of Formula Ib, 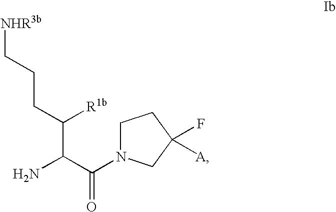
a prodrug thereof, or a pharmaceutically acceptable salt of the compound or the prodrug, wherein:
A is hydrogen or F;
R^{1b} is hydrogen, (C₁-C₈)alkyl, or (C₃-C₈)cycloalkyl;
R^{3b} is COR^{4b}, COOR^{5b}, CONR^{6b}R^{7b}, or SO₂NR^{6b}R^{7b};
R^{4b} and R^{5b} are (C₃-C₈)cycloalkyl, Het, or phenoxy(C₁-C₈)alkyl;
said phenoxy(C₁-C₈)alkyl includes a benzene ring optionally substituted with 1 to 3 of: (C₁-C₈)alkyl; (C₃-C₈)cycloalkyl; cyano; halo; (C₁-C₈)alkylsulfonyl; (C₁-C₈)alkylsulfonyloxy; phenyl(C₁-C₈)alkoxy; or phenyl, optionally substituted with 1 to 3 of (C₁-C₈)alkyl, halo, (C₁-C₈)alkoxy, cyano, hydroxy, trifluoromethyl, (C₁-C₈)alkylsulfonyl, (C₁-C₈)alkylsulfonyloxy, or phenyl(C₁-C₈)alkoxy; and,
said Het is (I) a heterocycle selected from: furanyl, dihydrofuranyl, tetrahydrofuranyl, pyranyl, dihydropyranyl, tetrahydropyranyl, thienyl, dihydrothienyl, tetrahydrothienyl or a benzo-fused analogue of said heterocycle; wherein each heterocycle is optionally substituted on carbon or nitrogen with 1 to 3 of: (A) (C₃-C₈)cycloalkyl; (B) (C₁-C₈)alkylsulfonyl; (C) (C₁-C₈)alkylsulfonyloxy; (D) phenoxy(C₁-C₈)alkyl; wherein said phenoxy group comprises a phenyl ring optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; (E) phenyl, optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; (F) (C₁-C₈)alkyl, (G) cyano, (H) halo, (I) phenyl(C₁-C₈)alkoxy; or (J) trifluoromethyl; or
(II) a heterocycle selected from: pyridyl, pyridazinyl, pyrimidyl, pyrazinyl, imidazolyl, pyrazolyl, thiazolyl, isothiazolyl, oxazolyl, isoxazolyl, triazolyl, or a benzo-fused analog of said Het; wherein each Het is independently substituted on carbon or nitrogen with 1 to 3 of: (A) (C₃-C₈)cycloalkyl; (B) (C₁-C₈)alkylsulfonyl; (C) (C₁-C₈)alkylsulfonyloxy; (D) phenoxy(C₁-C₈)alkyl; wherein said phenoxy group comprises a phenyl ring optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; (E) phenyl, optionally substituted with 1 to 3 of halo, cyano, hydroxy, trifluoromethyl, (C₁-C₈)alkyl, (C₁-C₈)alkoxy, (C₁-C₈)alkylsulfonyl, (C₁-C₈)alkylsulfonyloxy, or phenyl(C₁-C₈)alkoxy; wherein said Het is optionally substituted with 1 to 3 of (C₁-C₈)alkyl, cyano, halo, phenyl(C₁-C₈)alkoxy or trifluoromethyl; and
R^{6b} and R^{7b} are taken separately and independently and are (A) (C₃-C₈)cycloalkyl; (B) phenoxy(C₁-C₈)alkyl; (C) a five- or six-membered unsaturated, partially saturated or saturated heterocyclyl; wherein said heterocyclyl comprises 1 to 3 of: N; O; or S; and said heterocyclyl is substituted with 1 to 3 of (C₃-C₈)cycloalkyl, phenyl, (C₁-C₈)alkylsulfonyl, (C₁-C₈)alkylsulfonyloxy, or phenyl(C₁-C₈)alkoxy, or (D) a nine- or ten-membered fused heterocyclyl, wherein said fused heterocyclyl comprises 1 to 5 of, N, O, or S; and said fused heterocyclyl is substituted with 1 to 3 of, (C₃-C₈)cycloalkyl, phenyl, (C₁-C₈)alkylsulfonyl, (C₁-C₈)alkylsulfonyloxy, or phenyl(C₁-C₈)alkoxy.

15. A compound of claim 14 wherein R^{1b} is hydrogen.

16. A compound of claim 14 wherein R^{3b} is COR^{4b}.

17. A compound of claim 14 having an S configuration at the stereogenic carbon atom adjacent to the primary amine.

18. A pharmaceutical composition comprising a compound of claim 14 or 17, a prodrug or stereoisomer thereof, or a pharmaceutically acceptable salt of the compound, prodrug, or stereoisomer, and a pharmaceutically acceptable vehicle, carrier, or diluent.

19. A pharmaceutical composition comprising:
a. a compound of claim 14 or 17, a prodrug or stereoisomer thereof, or a pharmaceutically acceptable salt of the compound, prodrug, or stereoisomer;
b. a compound selected from insulin or an insulin analog; insulinotropin; a biguanide; an α₂-antagonist; an imidazoline; a glitazone; an aldose reductase inhibitor; a glycogen phosphorylase inhibitor; a sorbitol dehydrogenase inhibitor; a fatty acid oxidation inhibitor; an α-glucosidase inhibitor; a β-agonist; phosphodiesterase inhibitor; a lipid-lowering agent; an antiobesity agent; vanadate; a vanadium complex; a peroxovanadium complex; an amylin antagonist; a glucagon antagonist; a growth hormone secretagogue; a gluconeogenesis inhibitor; a somatostatin analog; an inhibitor of renal glucose; an antilipolytic agent; or a prodrug of said compound or a pharmaceutically acceptable salt of said compound or prodrug; and,
c. a pharmaceutically acceptable carrier, vehicle, or diluent.

20. A method of inhibiting dipeptidyl peptidase-IV in a mammal comprising administering to said mammal in need of such treatment a therapeutically effective amount of a compound of claim 14 or 17, a prodrug or stereoisomer thereof, or a pharmaceutically acceptable salt of the compound, prodrug, or stereoisomer, and a pharmaceutically acceptable vehicle, carrier, or diluent.

21. A method of treating a condition mediated by dipeptidyl peptidase-IV in a mammal comprising administering to said mammal in need of such treatment a therapeutically effective amount of a compound of claim 14 or 17, a prodrug or stereoisomer thereof, or a pharmaceutically acceptable salt of the compound, prodrug, or stereoisomer, and a pharmaceutically acceptable vehicle, carrier, or diluent.

22. The method of claim 21 wherein the condition is type 2 diabetes, metabolic syndrome, hyperglycemia, impaired glucose tolerance, glucosuria, metabolic acidosis, arthritis, cataracts, diabetic neuropathy, diabetic nephropathy, diabetic retinopathy, diabetic cardiomyopathy, type 1 diabetes, obesity, a diabetic condition exacerbated by obesity, hypertension, hyperlipidemia, atherosclerosis, osteoporosis, osteopenia, frailty, bone loss, bone fracture, acute coronary syndrome, infertility due to polycystic ovary syndrome, disease progression in type 2 diabetes, anxiety, depression, insomnia, chronic fatigue, epilepsy, an eating disorder, chronic pain, alcohol addiction, a disease associated with intestinal motility, an ulcer, irritable bowel syndrome, inflammatory bowel syndrome or short bowel syndrome.

23. The method of claim 22 wherein the condition is type 2 diabetes.

24. A method of identifying an insulin secretagogue agent for diabetes, comprising:
a. administering an agent comprising the compound of claim 1 or 14 to a fasted, diabetic KK/H1J mouse; and
b. assessing a response in the mouse to a subsequent oral glucose challenge,
wherein said agent may be identified as a treatment for Type 2 diabetes, metabolic syndrome, hyperglycemia, impaired glucose tolerance, glucosuria, metabolic acidosis, arthritis, cataracts, diabetic neuropathy, diabetic nephropathy, diabetic retinopathy, diabetic cardiomyopathy, Type 1 diabetes, obesity, conditions exacerbated by obesity, hypertension, hyperlipidemia, atherosclerosis, osteoporosis, osteopenia, frailty, bone loss, bone fracture, acute coronary syndrome, infertility due to polycystic ovary syndrome, to prevent disease progression in Type 2 diabetes or short bowel syndrome.

25. (S)-[5-amino-6-oxo-6-(3,3,4,4-tetrafluoro-pyrrolidin-1-yl)-hexyl-carbamic acid benzylester;
(S)-N-[5-amino-6-oxo-6-(3,3,4,4-tetrafluoro-pyrrolidin-1-yl)-hexyl]-3-fluoro-benzamide;
(S)-N-[5-amino-6-oxo-6-(3,3,4,4-tetrafluoro-pyrrolidin-1-yl)-hexyl]-3-cyano-benzamide;
(S)-quinoxaline-2-carboxylic acid [5-amino-6-oxo-6-(3,3,4,4-tetrafluoro-pyrrolidin-1-yl)-hexyl]-amide;
(S)-N-[5-amino-6-oxo-6-(3,3,4,4-tetrafluoro-pyrrolidin-1-yl)-hexyl]-2-phenoxy-acetamide;
(S)-3-(2-chloro-phenyl-5-methyl-isoxazole-4-carboxylic acid [5-amino-6-oxo-6-(3,3,4,4-tetrafluoro-pyrrolidin-1-yl)-hexyl]-amide;
(S)-N-[5-amino-6-oxo-6-(3,3,4,4-tetrafluoro-pyrrolidin-1-yl)-hexyl]-2-thiophen-2-yl-acetamide;
(S)-N-[5-amino-6-oxo-6-(3,3,4,4-tetrafluoro-pyrrolidin-1-yl)-hexyl]-N′,N′-dimethylsulfamide;
(S)-5-methyl-2-phenyl-oxazole-4-carboxylic acid [5-amino-6-oxo-6-(3,3-4,4-tetrafluoro-pyrrolidin-1-yl)-hexyl]-amide;
(S)-pyrazine-2-carboxylic acid [5-amino-6-oxo-6-(3,3,4,4-tetrafluoro-pyrrolidin-1-yl)-hexyl]-amide;
(S)-quinoxaline-2-carboxylic acid [5-amino-6-(3-fluoro-azetidin-1-yl)-6-oxo-hexyl]-amide;
(S)-3-methoxy-quinoxaline-2-carboxylic acid [5-amino-6-oxo-6-(3,3,4,4-tetrafluoro-pyrrolidin-1-yl)-hexyl]-amide;
(S)-5-methyl-2-phenyl-oxazole-4-carboxylic acid [5-amino-6-(3-fluoro-azetidin-1-yl)-6-oxo-hexyl]-amide;
(S)-5-methyl-2-phenyl-oxazole-4-carboxylic acid [5-amino-6-(3,3-difluoro-azetidin-1-yl)-6-oxo-hexyl]-amide;
(S)-quinoxaline-2-carboxylic acid [5-amino-6-(3,3-difluoro-azetidin-1-yl)-6-oxo-hexyl]-amide;
(S)-5-methyl-2-phenyl-2H-[1,2,3]triazole-4-carboxylic acid [5-amino-6-(3,3-difluoro-azetidin-1-yl)-6-oxo-hexyl]-amide;
(S)-5-methyl-2-phenyl-2H-[1,2,3]triazole-4-carboxylic acid [5-amino-6-(3-fluoro-azetidin-1-yl)-6-oxo-hexyl]-amide;
(S)-3-methyl-quinoxaline-2-carboxylic acid [5-amino-6-oxo-6-(3,3,4,4-tetrafluoro-pyrrolidin-1-yl)-hexyl]-amide;
(S)-3-methylquinoxaline-2-carboxylic acid [5-amino-6-(3-fluoro-azetidin-1-yl)-6-oxo-hexyl]-amide;
(S)-3-methyl-quinoxaline-2-carboxylic acid [5-amino-6-(3,3-difluoro-azetidin-1-yl)-6-oxo-hexyl]-amide;
(S)-5-methyl-2-phenyl-2H-[1,2,3]triazole4-carboxylic acid [5-amino-6-oxo-6-(3,3,4,4-tetrafluoro-pyrrolidin-1-yl)-hexyl]-amide;
(S)-6,7-dimethylquinoxaline-2-carboxylic acid [5-amino-6-(3-fluoro-azetidin-1-yl)-6-oxo-hexyl]-amide;
(S)-6,7-dimethylquinoxaline-2-carboxylic Acid [5-amino-6-(3,3-difluoro-azetid in-1-yl)-6-oxo-hexyl]-amide;
(S)-6,7-dichloro-quinoxaline-2-carboxylic acid [5-amino-6-(3-fluoro-azetidin-1-yl)-6-oxo-hexyl]-amide;
(S)-6,7-dichloro-quinoxaline-2-carboxylic Acid [5-amino-6-(3,3-difluoro-azetidin-1-yl)-6-oxo-hexyl]-amide;
(S)-6,7-difluoro-quinoxaline-2-carboxylic acid [5-amino-6-(3-fluoro-azetidin-1-yl)-6-oxo-hexyl]-amide;
(S)-6,7-difluoro-quinoxaiine-2-carboxylic acid [5-amino-6-(3,3-difluoro-azetidin-1-yl)-6-oxo-hexyl]-amide;
(S)-2-phenyl-oxazole-4-carboxylic acid [5-amino-6-(3,3-difluoro-azetidin-1-yl)-6-oxo-hexyl]-amide;
(S)-2-phenyl-2H-[1,2,3]triazole-4-carboxylic acid [5-Amino-6-(3-fluoro-azetidin-1-yl)-6-oxo-hexyl]-amide;
(S)-2-phenyl-2H-[1,2,3]triazole-4-carboxylic acid [5-amino-6-(3,3-difluoro-azetidin-1-yl)-6-oxo-hexyl]-amide;
(S)-2-phenyl-oxazole-4-carboxylic acid [5-amino-6-(3-fluoro-azetidin-1-yl)-6-oxo-hexyl]-amide trifluoroacetate;
(S)-1-[5-amino-6-oxo-6-(3,3,4,4-tetrafluoro-pyrrolidin-1-yl)-hexyl]-3-benzyl-urea; or,
(S)-4-benzyl-piperidine-1-carboxylic acid [5-amino-6-oxo-6-(3,3,4,4-tetrafluoro-pyrrolidin-1-yl)-hexyl]-amide;
or a pharmaceutically acceptable thereof.

26. (S)-quinoxaline-2-carboxylic Acid [5-Amino-6-oxo-6-(3,3,4,4-tetrafluoro pyrrolidin-1-yl)-hexyl]-amide;
(S)-N-[5-amino-6-oxo-6-(3,3,4,4-tetrafluoro-pyrrolidin-1-yl)-hexyl]-2-phenoxy-acetamide;
(S)-quinoxaline-2-carboxylic Acid [5-Amino-6-(3-fluoro-azetidin-1-yl)-6-oxo-hexyl]-amide;
(S)-5-methyl-2-phenyl-oxazole-4-carboxylic Acid [5-Amino-6-(3,3-difluoro-azetidin-1-yl)-6-oxo-hexyl]-amide, or
(S)-3-methyl-quinoxaline-2-carboxylic Acid [5-amino-6-oxo-6-(3,3,4,4-tetrafluoro-pyrrolidin-1-yl)-hexyl]-amide;
or a pharmaceutically acceptable thereof.

27. (S)-5-methyl-2-phenyl-2H-[1,2,3]triazole-4-carboxylic acid [5-amino-6-(3,3-difluoro-pyrrolidin-1-yl)-6-oxo-hexyl]-amide;
(S)-5-methyl-2-phenyl-oxazole-4-carboxylic acid [5-amino-6-(3,3-difluoro-pyrrolidin-1-yl)-6-oxo-hexyl]-amide;
(S)-2-(4-methoxy-phenyl)-5-methyl-oxazole-4-carboxylic acid [5-amino-6-(3,3-difluoro-pyrrolidin-1-yl)-6-oxo-hexyl]-amide;
(S) 2-phenyl-5-trifluoromethyl-oxazole-4-carboxylic acid [5-amino-6-(3,3-difluoro-pyrrolidin-1-yl)-6-oxo-hexyl]-amide;
(S)-N-[5-amino-(3,3-difluoro-pyrrolidin-1-yl)-6-oxo-6-hexyl]-2-phenoxy-acetamide;
(2RS)-2,3-dihydro-benzofuran-2-carboxylic acid [(5S)-5-amino-6-(3,3-difluoro-pyrrolidin-1-yl)-6-oxo-hexyl]-amide;
(S)-N-[5-amino-6-(3,3-difluoro-pyrrolidin-1-yl)-6-oxo-hexyl]-2-(4-fluoro-phenoxy)-acetamide;
(S)-2-phenyl-oxazole4-carboxylic acid [5-amino-6-(3,3-difluoro-pyrrolidin-1-yl)-6-oxo-hexyl]-amide;
(S)-2-phenyl-2-H-[1,2,3]triazole-4-carboxylic acid [5-amino-6-(3,3-difluoro-pyrrolidin-1-yl)-6-oxo-hexyl]-amide;
(S)-2-(4-chloro-phenyl)-5-methyl-2-H-[1,2,3]triazole4-carboxylic acid [5-amino-6-(3,3-difluoro-pyrrolidin-1-yl)-6-oxo-hexyl]-amide;or,
(S)-5-methyl-1-phenyl-1-H-pyrazole-4-carboxylic acid [5-amino-6-(3,3-difluoro-pyrrolidin-1-yl)-6-oxo-hexyl]-amide;
or a pharmaceutically acceptable thereof.
